(19) United States
(12) Patent Application Publication
(10) Pub. No.: US 2005/0203094 A1
(43) Pub. Date: Sep. 15, 2005

(54) 2-[5-(5-carbamimidoyl-1h-heteroaryl)]-6-hydroxybiphenyl-3-yl derivatives as factor viia inhibitors

(76) Inventors: Aleksandr Kolesnikov, San Franciso, CA (US); Roopa Rai, San Carlos, CA (US); Dvorak Shrader, Belmont, CA (US); Wendy B. Young, San Mateo, CA (US)

Correspondence Address: AXYS PHARMACEUTICALS, INC., 180 KIMBALL WAY, SOUTH SAN FRANCISCO, CA, 94080, US

(21) Appl. No.: 10/504,119

(22) PCT Filed: Feb. 12, 2003
(86) PCT No.: PCT/US03/04081

§ 371 Date: May 5, 2005

Related U.S. Application Data
(60) Provisional application No. 60/356,473, filed on Feb. 13, 2002.
(60) Provisional application No. 60/439,043, filed on Jan. 8, 2003.

(51) Int. Cl. A61K 31/52; A61K 31/498; A61K 31/4745; C07D 471/02

(52) U.S. Cl. 514/248; 514/249; 514/263.23; 514/303; 544/277; 544/330; 546/118

(57) ABSTRACT

The present invention provides 2-[5-(5-carbamimidoyl-1H-heteroaryl)]-6-hydroxy-biphenyl-3-yl derivatives of formula (I): I That are Factor VIIa and Xa inhibitors

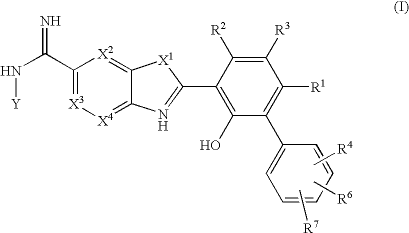

### BACKGROUND OF THE INVENTION

1. Field of Invention

The present invention relates to novel inhibitors of Factor VIIa and Factor Xa, in particular Factor VIIa, pharmaceutical compositions comprising these inhibitors and methods for using these inhibitors for treating or preventing thromboembolic disorders. Processes for preparing these inhibitors are also disclosed.

2. State of the Art

Thrombosis results from a complex sequence of biochemical events, known as the coagulation cascade. A triggering event in coagulation is the binding of the serine protease Factor VIIa found in the circulation to tissue factor (TF), a receptor which is found on the surface of blood vessels after damage or inflammation. Once bound to TF Factor VIIa catalyzes the formation of the serine protease Factor Xa, which subsequently forms the final protease in the cascade, thrombin.

The clinical manifestations of thrombosis range from acute myocardial infarction and unstable angina which occur in the key blood vessels of the heart to deep vein thrombosis (DVT) which is the formation of blood clots in lower extremities which often follows orthopedic surgery on the hip and knee, as well as general abdominal surgery and paralysis. Formation of DVT is a risk factor for the development of pulmonary embolism (PE) in which part of a blood clot formed in the lower extremities, breaks off and travels to the lung where it blocks the flow of blood. The unpredictable development of PE often leads to a fatal outcome. Thrombosis can also be generalized systemically with microclot formation occurring throughout the vascular system. This condition, known as disseminated intravascular coagulation (DIC), can be a consequence of certain viral diseases such as Ebola, certain cancers and sepsis. Severe DIC can lead to a dramatic reduction in the coagulation factors due to the excessive activation of the clotting response which may result in multiple organ failure, hemorrhage and death.

The formation or embolization of blood clots in the blood vessels of the brain is the key event resulting in ischemic stroke. Triggering factors that lead to stroke are atrial fibrillation or abnormal rhythm of the atria of the heart and atherosclerosis followed by thrombosis in the main artery leading from the heart to the brain, the carotid artery. Over 600,000 individuals suffer strokes each year in the U.S. Two-thirds of these stroke victims suffer some disability and one-third suffer permanent and severe disability. Accordingly, there is a need for antithrombotic agents for the treatment of a variety of thrombotic conditions. The present invention fulfills this and related needs.

### SUMMARY OF THE INVENTION

One aspect this invention provides a compound of Formula I: 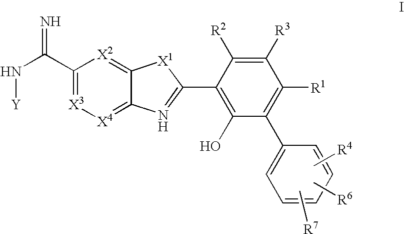
wherein:
X¹, X², X³ and X⁴ are independently —N— or —CR⁵—, wherein R⁵ is hydrogen, alkyl or halo, with the proviso that not more than three of X¹, X², X³ and X⁴ are —N—;
R¹ and R² independently are hydrogen, alkyl, hydroxyalkyl or halo;
R³ is:
   (i) hydroxyalkyl;
   (ii) hydroxyalkyl substituted with alkoxy, carboxy, oxo, tetrazol-5-yl or alkoxycarbonyl;
   (iii) carboxyalkyl substituted with tetrazol-5-yl, aryloxycarbonyl, alkylaminoalkoxycarbonyl, dialkylaminoalkoxycarbonyl, one to two alkoxy groups, one to four halo, amino, alkylamino, dialkylamino, oxo, —(OCH₂CH₂O)ₙ₁—OR (wherein n1 is an integer from 1 to 3 and R is hydrogen or alkyl) or —NR^{a}R^{b}, wherein R^{a} is hydrogen or alkyl and R^{b} is acyl or —SO₂R^{c}, wherein R^{c} is alkyl);
   (iv) cycloalkyl substituted with carboxy or carboxyalkyl;
   (v) carboxyalkyloxy or dicarboxyalkyloxy; or
   (vi) a group of formula (a), (b), (c), (d) or (e): 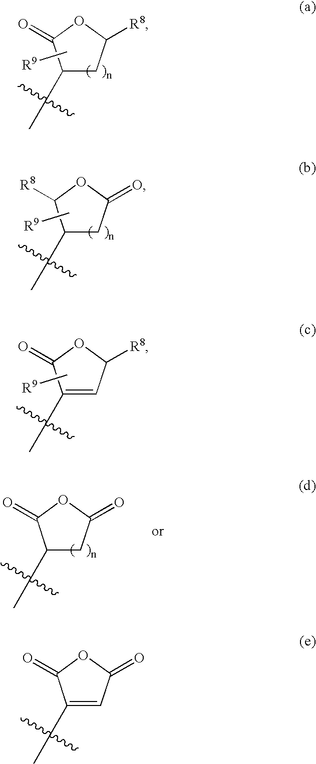
wherein:
   n is 1 or 2;
   R⁸ is hydrogen, alkyl, alkoxy or hydroxy; and
   R⁹ is hydrogen or alkyl;
R⁴ is hydrogen, alkyl, alkylthio, halo, hydroxy, hydroxyalkyl, alkoxy, aminosulfonyl, alkylaminosulfonyl, dialkylaminosulfonyl or nitro;
R⁶ is hydrogen, alkyl or halo;
R⁷ is hydrogen, alkyl, haloalkyl, cycloalkyl, alkylthio, halo, hydroxy, hydroxyalkyl, nitro, cyano, alkoxy, alkoxyalkyl, alkoxyalkyloxy, hydroxyalkyloxy, aminoalkyloxy, carboxyalkyloxy, aminocarbonylalkyloxy, haloalkoxy, carboxy, carboxyalkyl, alkoxycarbonyl, alkoxycarbonylalkyl, cyanoalkyl, alkylsulfonyl, alkylsulfonylalkyl, arylsulfonyl, heteroarylsulfonyl, carbamimidoyl, hydroxycarbamimidoyl, alkoxycarbamimidoyl, alkylsulfonylamino, alkylsulfonylaminoalkyl, alkoxysulfonylamino, alkoxysulfonylaminoalkyl, heterocycloalkylalkylaminocarbonyl, hydroxyalkoxyalkylaminocarbonyl, heterocycloalkylcarbonyl, heterocycloalkylcarbonylalkyl, heterocycloalkyl, heterocycloalkylalkyl, oxoheterocycloalkyl, oxoheterocycloalkylalkyl, heteroaryl, heteroaralkyl, ureido, alkylureido, dialkylureido, ureidoalkyl, alkylureidoalkyl, dialkylureidoalkyl, thioureido, thioureidoalkyl, acyldifluoromethyl, —COR¹² (wherein R¹² is alkyl, haloalkyl, hydroxyalkyl, alkoxyalkyl, oxoalkyl, dialkylaminocarbonylalkyl, alkoxycarbonylalkyl, cyanoalkyl or aminoalkyl), -(alkylene)-COR¹² (wherein R¹² is alkyl, haloalkyl, hydroxyalkyl, alkoxyalkyl or aminoalkyl), —CONR¹⁴R¹⁵ (wherein R¹⁴ is hydrogen or alkyl and R¹⁵ is hydrogen, alkyl, hydroxyalkyl, alkoxyalkyl, aryl, aralkyl, heteroaryl or heteroaralkyl), -(alkylene)-CONR¹⁶R¹⁷ (wherein R¹⁶ is hydrogen, alkyl or hydroxyalkyl and R¹⁷ is hydrogen, alkyl, hydroxyalkyl, alkoxyalkyl, aryl, aralkyl, heteroaryl or heteroaralkyl), —NR¹⁸R¹⁹ (wherein R¹⁸ is hydrogen or alkyl and R¹⁹ is hydrogen, alkyl, acyl, aryl, aralkyl, heteroaryl or heteroaralkyl), -(alkylene)-NR²⁰R²¹ (wherein R²⁰ is hydrogen, alkyl or hydroxyalkyl and R²¹ is hydrogen, alkyl, acyl, alkoxycarbonyl, hydroxyalkyl, alkoxyalkyl, aryl, aralkyl, heteroaryl or heteroaralkyl), —SO₂NR²²R²³ (wherein R²² is hydrogen or alkyl and R²³ is hydrogen, alkyl, aryl, aralkyl, heteroaryl or heteroaralkyl or R²² and R²³ together with the nitrogen atom to which they are attached from heterocycloamino), -(alkylene)-SO₂NR²⁴R²⁵ (wherein R²⁴ is hydrogen or alkyl and R²⁵ is hydrogen, alkyl, aryl, aralkyl, heteroaryl or heteroaralkyl or R²⁴ and R²⁵ together with the nitrogen atom to which they are attached from heterocycloamino), —NR²⁶SO₂NR²⁷R²⁸ (wherein R²⁶ and R²⁷ are independently hydrogen or alkyl and R²⁸ is hydrogen, alkyl, aryl, aralkyl, heteroaryl or heteroaralkyl or R²⁷ and R²⁸ together with the nitrogen atom to which they are attached from heterocycloamino), -(alkylene)-NR²⁹SO₂NR³⁰R³¹ (wherein R²⁹ and R³⁰ are independently hydrogen or alkyl and R³¹ is hydrogen, alkyl, aryl, aralkyl, heteroaryl or heteroaralkyl or R³⁰ and R³¹ together with the nitrogen atom to which they are attached from heterocycloamino), —CONH-(alkylene)-NR³²R³³ (wherein R³² is hydrogen or alkyl and R³³ is alkyl) or aralkyl; and
Y is hydrogen, hydroxy, alkoxy, haloalkoxy, haloalkoxycarbonyl, —C(O)R³⁵ (wherein R³⁵ is alkyl, aryl, haloalkyl, or cyanoalkyl) or —C(O)OR³⁶ (wherein R³⁶ is alkyl, hydroxyalkyl, alkoxyalkyl, alkoxycarbonylalkyl, acyl, aryl or haloalkyl); and individual stereoisomers or mixture of stereoisomers; or a pharmaceutically acceptable salt thereof; provided that when R³ is: (i) carboxyalkyl substituted with aryloxycarbonyl, one to two alkoxy groups, one to four halo, amino, alkylamino, dialkylamino or oxo or (ii) cycloalkyl substituted with carboxy and R⁷ is hydrogen, alkyl, haloalkyl, halo, nitro, alkoxy, haloalkyl, carboxy, alkoxycarbonyl, —NR¹⁸R¹⁹ (wherein R¹⁸ is hydrogen or alkyl and R¹⁹ is hydrogen, alkyl, aryl or aralkyl), pyrrolidinylcarbonyl, —SO₂NR²²R²³ (wherein R²² and R²³ are alkyl), carbamimidoyl, alkylsulfonylamino, alkylthio, ureido, —NHC(S)NH₂ or heterocycloamino, then R⁴ is hydroxy or hydroxyalkyl.

Another aspect of this invention provides a compound of Formula I: wherein:
X¹, X², X³ and X⁴ are independently —N— or —CR⁵—, wherein R⁵ is hydrogen, alkyl or halo, with the proviso that not more than three of X¹, X², X³ and X⁴ are —N—;
Y is hydrogen, hydroxy, alkoxy, haloalkoxy or haloalkoxycarbonyl;
R¹ and R² independently are hydrogen, alkyl, hydroxyalkyl or halo;
R³ is:
   (i) hydroxyalkyl;
   (ii) hydroxyalkyl substituted with carboxy, oxo, tetrazol-5-yl or alkoxycarbonyl;
   (iii) carboxyalkyl substituted with tetrazol-5-yl, aryloxycarbonyl, alkylaminoalkoxycarbonyl, dialkylaminoalkoxycarbonyl or one to two alkoxy groups; or
   (iv) a group of formula (a), (b), (c), (d) or (e): 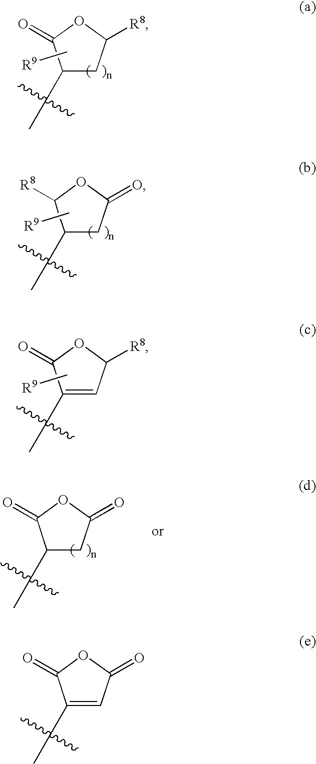
wherein:
   n is 1 or 2;
   R⁸ is hydrogen, alkyl, alkoxy or hydroxy; and
   R⁹ is hydrogen or alkyl;
R⁴ is hydrogen, alkyl, alkylthio, halo, hydroxy, hydroxyalkyl, alkoxy or nitro;
R⁶ is hydrogen, alkyl or halo; and

R⁷ is hydrogen, alkyl, halo, hydroxy, nitro, cyano, alkoxy, haloalkyl, haloalkoxy, —COR¹⁰ (wherein R¹⁰ is alkyl), aminocarbonyl, hydroxyalkyl, carboxy, carboxyalkyl, amino, alkylamino, dialkylamino, heterocycloalkylalkylaminocarbonyl, cyanoalkyl, aminocarbonylalkyl, alkoxyalkyl, hydroxyalkoxyalkylaminocarbonyl, heterocycloalkylcarbonyl, heterocycloalkylalkyl, oxoheterocycloalkylalkyl, carbamimidoyl, aminosulfonylamino, alkylaminosulfonylamino, alkylsulfonylamino, alkylthio, aminoalkyl, ureidoalkyl, heteroaryl, ureido or thioureido; and individual isomers or mixture of isomers; or a pharmaceutically acceptable salt thereof.

Another aspect of this invention is directed to a pharmaceutical composition comprising a pharmaceutically acceptable carrier and a therapeutically effective amount of a compound of Formula I; or a pharmaceutically acceptable salt thereof.

Another aspect of this invention is directed to a method of treating a disease in an animal mediated by Factors VIIa and/or Xa, preferably VIIa, which method comprises administering to said animal a pharmaceutical composition comprising a pharmaceutically acceptable carrier and a therapeutically effective amount of a compound of Formula I or a pharmaceutically acceptable salt thereof. Preferably, the disorder is a thromboembolic disorder.

Another of this invention is directed to an intermediate of Formula II: 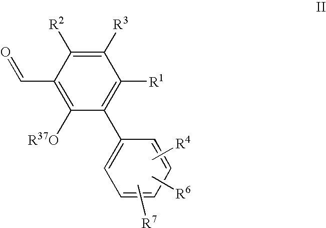
wherein:
R¹ and R² independently are hydrogen, alkyl, hydroxyalkyl or halo;
R³ is:
   (vii) hydroxyalkyl;
   (viii) hydroxyalkyl substituted with alkoxy, carboxy, oxo, tetrazol-5-yl or alkoxycarbonyl;
   (ix) carboxyalkyl substituted with tetrazol-5-yl, aryloxycarbonyl, alkylaminoalkoxycarbonyl, dialkylaminoalkoxycarbonyl, one to two alkoxy groups, one to four halo, amino, alkylamino, dialkylamino, oxo, —(OCH₂CH₂O)ₙ₁—OR (wherein n1 is an integer from 1 to 3 and R is hydrogen or alkyl) or —NR^{a}R^{b}, wherein R^{a} is hydrogen or alkyl and R^{b} is acyl or —SO₂R^{c}, wherein R^{c} is alkyl);
   (x) cycloalkyl substituted with carboxy or carboxyalkyl;
   (xi) carboxyalkyloxy or dicarboxyalkyloxy; or
   (xii) a group of formula (a), (b), (c), (d) or (e): 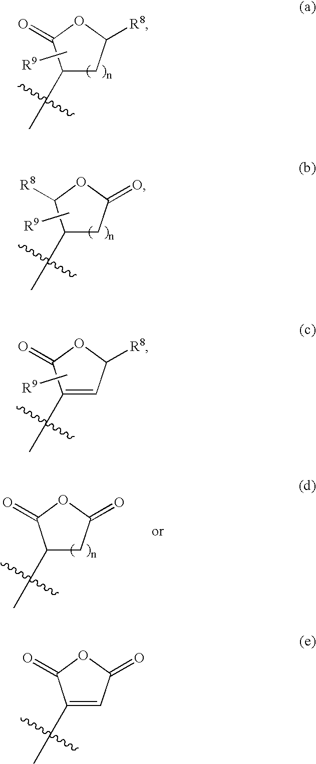
wherein:
   n is 1 or 2;
   R⁸ is hydrogen, alkyl, alkoxy or hydroxy; and
   R⁹ is hydrogen or alkyl;
R⁴ is hydrogen, alkyl, alkylthio, halo, hydroxy, hydroxyalkyl, alkoxy, aminosulfonyl, alkylaminosulfonyl, dialkylaminosulfonyl or nitro;
R⁶ is hydrogen, alkyl or halo;
R⁷ is hydrogen, alkyl, haloalkyl, cycloalkyl, alkylthio, halo, hydroxy, hydroxyalkyl, nitro, cyano, alkoxy, alkoxyalkyl, alkoxyalkyloxy, hydroxyalkyloxy, aminoalkyloxy, carboxyalkyloxy, aminocarbonylalkyloxy, haloalkoxy, carboxy, carboxyalkyl, alkoxycarbonyl, alkoxycarbonylalkyl, cyanoalkyl, alkylsulfonyl, alkylsulfonylalkyl, arylsulfonyl, heteroarylsulfonyl, carbamimidoyl, hydroxycarbamimidoyl, alkoxycarbamimidoyl, alkylsulfonylamino, alkylsulfonylaminoalkyl, alkoxysulfonylamino, alkoxysulfonylaminoalkyl, heterocycloalkylalkylaminocarbonyl, hydroxyalkoxyalkylaminocarbonyl, heterocycloalkylcarbonyl, heterocycloalkylcarbonylalkyl, heterocycloalkyl, heterocycloalkylalkyl, oxoheterocycloalkyl, oxoheterocycloalkylalkyl, heteroaryl, heteroaralkyl, ureido, alkylureido, dialkylureido, ureidoalkyl, alkylureidoalkyl, dialkylureidoalkyl, thioureido, thioureidoalkyl, acyldifluoromethyl, —COR¹² (wherein R¹² is alkyl, haloalkyl, hydroxyalkyl, alkoxyalkyl, oxoalkyl, dialkylaminocarbonylalkyl, alkoxycarbonylalkyl, cyanoalkyl or aminoalkyl), -(alkylene)-COR¹² (wherein R¹² is alkyl, haloalkyl, hydroxyalkyl, alkoxyalkyl or aminoalkyl), —CONR¹⁴R¹⁵ (wherein R¹⁴ is hydrogen or alkyl and R¹⁵ is hydrogen, alkyl, hydroxyalkyl, alkoxyalkyl, aryl, aralkyl, heteroaryl or heteroaralkyl), -(alkylene)-CONR¹⁶R¹⁷ (wherein R¹⁶ is hydrogen, alkyl or hydroxyalkyl and R¹⁷ is hydrogen, alkyl, hydroxyalkyl, alkoxyalkyl, aryl, aralkyl, heteroaryl or heteroaralkyl), —NR¹⁸R¹⁹ (wherein R¹⁸ is hydrogen or alkyl and R¹⁹ is hydrogen, alkyl, acyl, aryl, aralkyl, heteroaryl or heteroaralkyl), -(alkylene)-NR²⁰R²¹ (wherein R²⁰ is hydrogen, alkyl or hydroxyalkyl and R²¹ is hydrogen, alkyl, acyl, alkoxycarbonyl, hydroxyalkyl, alkoxyalkyl, aryl, aralkyl, heteroaryl or heteroaralkyl), —SO₂NR²²R²³ (wherein R²² is hydrogen or alkyl and R²³ is hydrogen, alkyl, aryl, aralkyl, heteroaryl or heteroaralkyl or R²² and R²³ together with the nitrogen atom to which they are attached from heterocycloamino), -(alkylene)-SO₂NR²⁴R²⁵ (wherein R²⁴ is hydrogen or alkyl and R²⁵ is hydrogen, alkyl, aryl, aralkyl, heteroaryl or heteroaralkyl or R²⁴ and R²⁵ together with the nitrogen atom to which they are attached from heterocycloamino), —NR²⁶SO₂NR²⁷R²⁸ (wherein R²⁶ and R²⁷ are independently hydrogen or alkyl and R²⁸ is hydrogen, alkyl, aryl, aralkyl, heteroaryl or heteroaralkyl or R²⁷ and R²⁸ together with the nitrogen atom to which they are attached from heterocycloamino), -(alkylene)-NR²⁹SO₂NR³⁰R³¹ (wherein R²⁹ and R³⁰ are independently hydrogen or alkyl and R³¹ is hydrogen, alkyl, aryl, aralkyl, heteroaryl or heteroaralkyl or R³⁰ and R³¹ together with the nitrogen atom to which they are attached from heterocycloamino), —CONH-(alkylene)-NR³²R³³ (wherein R³² is hydrogen or alkyl and R³³ is alkyl) or aralkyl; and
Y is hydrogen, hydroxy, alkoxy, haloalkoxy, haloalkoxycarbonyl, —C(O)R³⁵ (wherein R³⁵ is alkyl, aryl, haloalkyl, or cyanoalkyl) or —C(O)OR³⁶ (wherein R³⁶ is alkyl, hydroxyalkyl, alkoxyalkyl, alkoxycarbonylalkyl, acyl, aryl or haloalkyl); and individual stereoisomers or mixture of stereoisomers; or a pharmaceutically acceptable salt thereof; provided that when R³ is: (i) carboxyalkyl substituted with aryloxycarbonyl, one to two alkoxy groups, one to four halo, amino, alkylamino, dialkylamino or oxo or (ii) cycloalkyl substituted with carboxy and R⁷ is hydrogen, alkyl, haloalkyl, halo, nitro, alkoxy, haloalkyl, carboxy, alkoxycarbonyl, —NR¹⁸R¹⁹ (wherein R¹⁸ is hydrogen or alkyl and R¹⁹ is hydrogen, alkyl, aryl or aralkyl), pyrrolidinylcarbonyl, —SO₂NR²²R²³ (wherein R²² and R²³ are alkyl), carbamimidoyl, alkylsulfonylamino, alkylthio, ureido, —NHC(S)NH₂ or heterocycloamino, then R⁴ is hydroxy or hydroxyalkyl.

Another aspect of this invention is directed to a process for preparing a compound of Formula I wherein X¹ is —N—, comprising reacting a compound of Formula II with a compound of Formula III: 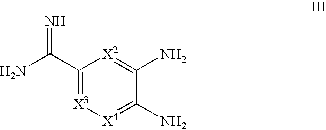
(i) optionally modifying any of the R¹, R², R⁴, R⁶, R⁷, R⁸ or R¹⁰ groups;
(ii) optionally preparing an acid addition salt; and
(iii) optionally preparing a free base.

Further aspects of this invention are found in U.S. Provisional Patent Application Nos. 60/356,473, filed on Feb. 13, 2002, and 60/439,043, filed on Jan. 8, 2003, both disclosures of which are incorporated herein by reference in their entirety.

### DETAILED DESCRIPTION OF THE INVENTION

Definitions

The following terms, as used in the present specification and claims, are intended to have the meaning as defined below, unless indicated otherwise.

“Acyl” means a radical —COR′ wherein R′ is alkyl, alkoxy, haloalkyl, aminoalkyl, hydroxyalkyl or alkoxyalkyl, as such terms are defined herein, e.g., acetyl, trifluoroacetyl, hydroxymethylcarbonyl, and the like.

“Alkoxy” means a radical —OR wherein R is alkyl, as defined herein, e.g., methoxy, ethoxy, propoxy, 2-propoxy, n-, iso- or tert-butoxy, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined herein, e.g., 2-methoxyethyl, 1-, 2- or 3-methoxypropyl, 2-ethoxyethyl, and the like.

“Alkoxyalkyloxy” means a radical —OR wherein R is alkoxyalkyl, as defined herein, e.g., 2-methoxyethyloxy, 1-, 2- or 3-methoxypropyloxy, 2-ethoxyethyloxy, and the like.

“Alkoxycarbamimidoyl” means a radical —C(═NH)NHOR or —C(═NOR)NH₂ wherein R is alkyl, as defined herein, e.g., methoxycarbamimidoyl.

“Alkoxycarbonyl” means a radical —C(O)R wherein R is alkoxy, as defined herein, e.g., methoxycarbonyl, ethoxycarbonyl, propoxycarbonyl, 2-propoxycarbonyl, n-, iso- or tert-butoxycarbonyl, and the like.

“Alkoxycarbonylalkyl” means a radical -(alkylene)-COOR wherein R is alkyl, in which alkyl and alkylene are as defined herein, e.g., methoxycarbonylmethyl, ethoxycarbonylmethyl, and the like.

“Alkoxysulfonylamino” means a radical —NHSO₂R wherein R is alkoxy, as defined herein, e.g., methoxysulfonylamino, ethoxysulfonylamino, and the like.

“Alkoxysulfonylaminoalkyl” means a radical -(alkylene)-NHSO₂R wherein R is alkoxy, in which alkoxy and alkylene are as defined herein, e.g., methoxysulfonylaminomethyl, ethoxysulfonylaminomethyl, and the like.

“Alkyl”, either when occurring by itself or as part of an alkyl substituted radical (e.g., alkylthio, and the like), means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like. “Alkyl”, when occurring as part of a substituted alkyl radical (e.g., alkoxyalkyl, alkoxysulfonylaminoalkyl, aminoalkyl, and the like), means alkylene, as defined herein.

“Alkylene” means a linear saturated divalent hydrocarbon radical of one to six carbon atoms or a branched saturated divalent hydrocarbon radical of three to six carbon atoms e.g., methylene (—CH₂—), ethylene (—CH₂CH₂—), propylene (—CH₂CH₂CH₂—), 1-methylpropylene (—CH₂(CH₃)CH₂CH₂—), 2-methylpropylene (—CH₂CH₂(CH₃)CH₂—), butylene (—CH₂CH₂CH₂CH₂—), pentylene, (—CH₂CH₂CH₂CH₂CH₂—), and the like.

“Alkylthio” means a radical —SR wherein R is alkyl, as defined herein, e.g., methylthio, ethylthio, propylthio (including all isomeric forms), butylthio (including all isomeric forms), and the like.

“Alkylamino” means a radical —NHR wherein R is alkyl, as defined herein, (e.g., methylamino, ethylamino, n- or iso-propylamino, n-, iso- or tert-butylamino, methylamino-N-oxide, and the like) and the term, either when occurring by itself or as part of an alkylamino substituted radical (e.g., alkylaminosulfonyl, alkylaminosulfonylamino, and the like), is meant to include the N-oxide derivative, i.e., —NR→O, or the protected derivatives thereof.

“Alkylaminosulfonyl” means a radical —SO₂NHR wherein R is alkyl, as defined herein, e.g., methylaminosulfonyl, ethylamino-sulfonyl, and the like.

“Alkylaminosulfonylamino” means a radical —NHSO₂NHR′ wherein R′ is alkyl, as defined herein, or an N-oxide derivative, or a protected derivative thereof, e.g., ethylaminosulfonylamino, ethylaminosulfonylamino, n- or iso-propylaminosulfonylamino, and the like.

“Alkylsulfonylamino” means a radical —NHSO₂R wherein R is alkyl, as defined herein, or an N-oxide derivative, or a protected derivative thereof, e.g., methylsulfonylamino, ethylsulfonylamino, n- or iso-propylsulfonylamino, and the like.

“Alkylsulfonylaminoalkyl” means a radical -(alkylene)-NHSO₂R wherein R is alkyl, in which alkyl and alkylene are as defined herein, e.g., methylsulfonylaminomethyl, ethylsulfonylaminomethyl, n- or iso-propylsulfonylaminoethyl, and the like.

“Alkylsulfonyl” means a radical —SO₂R wherein R is alkyl, as defined herein, e.g., methylsulfonyl, ethylsulfonyl, n- or iso-propylsulfonyl, and the like.

“Alkylsulfonylalkyl” means a radical -(alkylene)-SO₂R wherein R is alkyl, in which alkyl and alkylene are as defined herein, e.g., methylsulfonylmethyl, ethylsulfonylmethyl, n- or iso-propylsulfonylethyl, and the like.

“Alkylureido” means a radical —NRCONHR′ wherein R is hydrogen or alkyl, as defined herein, and R′ is alkyl, as defined herein, e.g., methylureidomethyl, and the like.

“Alkylureidoalkyl” means a radical -(alkylene)-NRCONHR′ wherein R is hydrogen or alkyl and R′ is alkyl, in which alkyl and alkylene are as defined herein, e.g., methylureidomethyl, and the like.

“Amino” means a radical —NH₂ and the term, either when occurring by itself or as part of an amino substituted radical (e.g., aminoalkyl, aminoalkyloxy, aminosulfonylamino, and the like) is meant to include the N-oxide derivative, i.e., —NH→O, or the protected derivatives thereof (e.g, tert-butoxycarbonylamino, benzyloxycarbonylamino, and the like).

“Aminoalkyl” means a linear monovalent hydrocarbon radical of two to six carbon atoms or a branched monovalent hydrocarbon radical of three to six carbons substituted with at least one, preferably one or two, —NRR′ wherein R and R′ are independently selected from hydrogen, alkyl, or —COR^{a} wherein R^{a} is alkyl, or an N-oxide derivative, or a protected derivative thereof, e.g., aminomethyl, methylaminoethyl, 2-ethylamino-2-methylethyl, 1,3-diaminopropyl, dimethylaminomethyl, diethylaminoethyl, acetylaminopropyl, and the like.

“Aminoalkyloxy” means a radical —OR wherein R is aminoalkyl, as defined herein, e.g., 2-aminoethyloxy, 1-, 2- or 3-methylaminopropyloxy, and the like.

“Aminosulfonylamino” means a radical —NHSO₂NH₂, or an N-oxide derivative, or a protected derivative thereof.

“Aminocarbonyl” means a radical —CONH₂, or an N-oxide derivative, or a protected derivative thereof.

“Aminocarbonylalkyl” means a radical -(alkylene)-CONH₂, in which alkylene is as defined herein, or an N-oxide derivative, or a protected derivative thereof, e.g., aminocarbonylmethyl, aminocarbonylethyl, 1-, 2- or 3-aminocarbonylpropyl, and the like.

“Aminocarbonylalkyloxy” means a radical —O-alkylene-CONRR″ wherein R and R′ are independently hydrogen or alkyl, in which alkyl and alkylene are as defined herein, e.g., 2-aminocarbonylethyloxy, aminocarbonylmethyloxy, and the like.

“Aryl” means a monovalent monocyclic or bicyclic aromatic hydrocarbon radical of 6 to 12 ring atoms, optionally substituted independently with one or more substituents, preferably one or two substituents, selected from alkyl, haloalkyl, alkoxy, alkylthio, halo, nitro, —COR (wherein R is alkyl), cyano, amino, alkylamino, dialkylamino, hydroxy, carboxy or —COOR wherein R is alkyl. Representative examples include, but are not limited to, phenyl, biphenyl, 1-naphthyl, and 2-naphthyl, and the substituted derivatives thereof.

“Aralkyl” means a radical -(alkylene)-R wherein R is an aryl group, in which aryl and alkylene are as defined herein e.g., benzyl, phenylethyl, 3-(3-chlorophenyl)-2-methylpentyl, and the like.

“Arylsulfonyl” means a radical —SO₂R wherein R is aryl, as defined herein, e.g., phenylsulfonyl, and the like.

“Aryloxycarbonyl” means a radical —C(O)OR wherein R is aryl, as defined herein e.g., phenoxycarbonyl, and the like.

“Alkylaminoalkoxycarbonyl” means a radical —C(O)O(alkylene)-R wherein R is alkylamino, in which alkylamino and alkylene are as defined herein e.g., methylaminoethyloxycarbonyl, and the like.

“Biphenylyl moiety” means that moiety comprising the compounds of Formula I having the following formula: 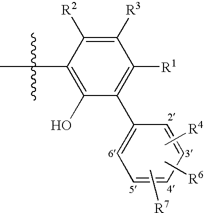
in which each R¹, R², R³, R⁴, R⁶ and R⁷ are as defined in the Summary of the Invention.

“Cycloalkyl” means a cyclic saturated monovalent hydrocarbon radical of three to six carbon atoms, e.g., cyclopropyl, cyclobutyl, and the like.

“Carboxyalkyl” means a radical -(alkylene)-COOH, in which alkylene is as defined herein, e.g., carboxymethyl, carboxyethyl, 1-, 2- or 3-carboxypropyl, and the like.

“Carboxyalkyloxy” means a radical —O-(alkylene)-COOH, in which alkylene is as defined herein, e.g., carboxymethyloxy, carboxyethyloxy, and the like.

“Carbamimidoyl” means a radical —C(═NH)NH₂, or an N-oxide derivative, or a protected derivative thereof.

“Cyanoalkyl” means a radical -(alkylene)-CN, in which alkylene is as defined herein, e.g., cyanomethyl, cyanoethyl, cyanopropyl, and the like.

“Dialkylamino” means a radical —NRR′ wherein R and R′ are independently alkyl, as defined herein, e.g., dimethylamino, diethylamino, methylpropylamino, methylethylamino, n-, iso-, or tert-butylamino, and the like.

“Dialkylaminosulfonylamino” means a radical —NHSO₂NRR′ wherein R and R′ are alkyl, as defined herein, e.g., dimethylaminosulfonylamino, diethylaminosulfonylamino, di-n- or iso-propylaminosulfonyl-amino, and the like.

“Dialkylaminoalkoxycarbonyl” means a radical —C(O)O(alkylene)-R wherein R is dialkylamino, as defined herein, e.g., dimethylaminoethyloxycarbonyl, and the like.

“Dialkylaminosulfonyl” means a radical —SO₂NRR′ wherein R and R′ are independently alkyl, as defined herein, e.g., dimethylaminosulfonyl, methylethylaminosulfonyl, and the like.

“Dicarboxyalkyloxy” means a radical —OR wherein R is alkyl, as defined herein, that is substituted with two carboxy groups, e.g., 2,2-dicarboxyethyloxy, and the like.

“Dialkylureido” means a radical —NRCONR′R″ wherein R is hydrogen or alkyl and R′ and R″ are independently alkyl, as defined herein, e.g., dimethylureido, and the like.

“Dialkylureidoalkyl” means a radical -(alkylene)-NRCONR′R″ wherein R is hydrogen or alkyl and R′ and R″ are independently alkyl, in which alkyl and alkylene are as defined herein, e.g., dimethylureidomethyl, and the like.

“Halo” means fluoro, chloro, bromo or iodo.

“Haloalkyl” means alkyl substituted with one or more halogen atoms, preferably one to three halogen atoms, including those substituted with different halogens, e.g., —CH₂Cl, —CF₃, —CHF₂, and the like.

“Haloalkoxy” means a radical —OR wherein R is haloalkyl as defined herein, e.g., —OCH₂Cl, —OCF₃, —OCHF₂, and the like.

“Haloalkoxycarbonyl” means a radical —C(O)R wherein R is haloalkoxy, as defined herein, e.g., —C(O)O(CH₂)₂CCl₃, —C(O)OCF₃, —OCHF₂, and the like.

“Heterocycloalkyl” means a saturated monovalent cyclic group of 3 to 8 ring atoms in which one or two ring atoms are heteroatoms selected from N, O, or S(O)n, wherein n is an integer from 0 to 2, the remaining ring atoms being C. The heterocycloalkyl ring may be substituted with one or more substituents, preferably one or two substituents, independently selected from alkyl, aryl, heteroaryl, aralkyl, heteroaralkyl, haloalkyl, halo, cyano, carboxy, or —COOR wherein R is alkyl as define above. More specifically the term heterocycloalkyl; includes, but is not limited to, pyrrolidino, piperidino, morpholino, piperazino, tetrahydropyranyl, and thiomorpholino, and the substituted derivatives thereof; and the N-oxide and/or protected derivatives thereof.

“Heterocycloalkylcarbonyl” means a radical —COR wherein R is heterocycloalkyl, as defined herein. More specifically the term heterocycloalkylcarbonyl includes, but is not limited to, 1-pyrrolidinocarbonyl, 1-piperidinocarbonyl, 4-morpholinocarbonyl, 1-piperazinocarbonyl, 2-tetrahydropyranylcarbonyl, and 4-thiomorpholinocarbonyl, and the substituted derivatives thereof.

“Heterocycloalkylcarbonylalkyl” means a radical -(alkylene)-COR wherein R is heterocycloalkyl, in which heterocycloalkyl and alkylene are as defined herein. More specifically the term heterocycloalkylcarbonyl includes, but is not limited to, 1-pyrrolidinocarbonylmethyl, 1-piperidinocarbonylmethyl, 4-morpholinocarbonylethyl, 1-piperazinocarbonylmethyl, and the substituted derivatives thereof.

“Heterocycloalkylalkyl” means a radical -(alkylene)-R wherein R is heterocycloalkyl, in which heterocycloalkyl and alkylene are as defined herein. More specifically the term heterocycloalkylalkyl includes, but is not limited to, 1-pyrrolidinomethyl, 1-piperidinomethyl, 4-morpholinoethyl, 1-piperazinoethyl, and the substituted derivatives thereof.

“Heterocycloalkylalkylaminocarbonyl” means a radical —CONH-(alkylene)-R wherein R is heterocycloalkyl, in which heterocycloalkyl and alkylene are as defined herein. More specifically the term heterocycloalkylalkylamino-carbonyl includes, but is not limited to, 1-pyrrolidinoethylaminocarbonyl, 1-piperidinoethylaminocarbonyl, 4-morpholinoethylcarbonyl, 1-piperazinoethylaminocarbonyl, and 4-thiomorpholinopropylaminocarbonyl, and the substituted derivatives thereof.

“Heteroaryl” means a monovalent monocyclic or bicyclic aromatic radical of 5 to 10 ring atoms containing one or more, preferably one or two ring heteroatoms selected from N, O, or S, the remaining ring atoms being carbon. The heteroaryl ring is optionally substituted with one or more substituents, preferably one or two substituents, independently selected from alkyl, haloalkyl, alkoxy, alkylthio, halo, nitro, cyano, amino, alkyl or dialkylamino, hydroxy, carboxy, or —COOR wherein R is alkyl as define above. More specifically the term heteroaryl includes, but is not limited to, pyridyl, pyrrolyl, imidazolyl, thienyl, furanyl, indolyl, quinolyl, pyrazine, pyrimidine, pyradizine, oxazole, isooxazolyl, benzoxazole, quinoline, isoquinoline, benzopyranyl, and thiazolyl, and the substituted derivatives thereof; and the N-oxide and/or protected derivatives thereof.

“Heteroaralkyl” means a radical -(alkylene)-R wherein R is a heteroaryl, in which heteroaryl and alkylene are as defined herein, e.g., pyridylmethyl, furanylmethyl, indolylmethyl, pyrimidinylmethyl, and the like.

“Heteroarylsulfonyl” means a radical —SO₂R wherein R is heteroaryl, as defined herein, e.g., pyridylsulfonyl, furanylsulfonyl, and the like.

“Heterocycloamino” means a saturated or unsaturated monovalent cyclic group of 3 to 8 ring atoms in which one or two ring atoms are heteroatoms selected from N, O, or S(O)ₙ, wherein n is an integer from 0 to 2, the remaining ring atoms being C provided that at least one of the heteroatom is nitrogen and wherein one or two carbon atoms are optionally replace by a carbonyl group. The heterocycloamino ring is optionally substituted with one or more substituents, preferably one or two substituents, independently selected from alkyl, hydroxy, hydroxyalkyl, alkoxy, alkoxyalkyl, halo, haloalkyl, aryl, heteroaryl, aralkyl, heteroaralkyl, haloalkyl, halo, cyano, carboxy, —CONR^{a}R^{b} (wherein R^{a} and R^{b} are independently hydrogen or alkyl) or —COOR wherein R is alkyl as define above. More specifically the term heterocycloamino includes, but is not limited to, pyrrolidino, piperidino, piperazino, and thiomorpholino, and the substituted derivatives thereof.

“Hydroxy” means the radical —OH and, unless indicated otherwise, the term, either when occurring by itself as a part of a hydroxy substituted radical (e.g., hydroxyalkyl, hydroxyalkyloxy, hydroxyalkoxyalkylaminocarbonyl, and the like), is meant to include the protected derivatives thereof (e.g., 2-methoxyethoxymethoxy, methoxymethoxy, acetoxy, and the like). Accordingly, suitable hydroxy protecting groups include 2-methoxyethoxymethyl, methoxymethyl, acetyl, and the like).

“Hydroxyalkyl” means a linear monovalent hydrocarbon radical of one to six carbon atoms or a branched monovalent hydrocarbon radical of three to six carbons substituted with one or two hydroxy groups, as defined herein, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl. Preferably, 2-hydroxy-1-hydroxymethylethyl or 3-hydroxy-1-hydroxymethylpropyl.

“Hydroxyalkyloxy” means a radical —OR wherein R is hydroxyalkyl, as defined herein, e.g., 2-hydroxyethyloxy, 3-hydroxypropyloxy, and the like.

“Hydroxyalkoxyalkylaminocarbonyl” means a radical —CONH-(alkylene)-O-(alkylene)OH wherein alkylene is as defined herein, e.g., —CONH—(CH₂)₂—O—(CH₂)₂OH and the like.

“Hydroxycarbamimidoyl” means a radical —C(═NH)NHOH or —C(═NOH)NH₂.

The present invention also includes the prodrugs of compounds of Formula I. The term prodrug is intended to represent covalently bonded carriers, which are capable of releasing the active ingredient of Formula I when the prodrug is administered to a mammalian subject. Release of the active ingredient occurs in vivo. In fact, some of the compounds of Formula I may act as prodrugs, i.e., release an active compound in vivo. Such prodrugs are within the scope of this invention. Prodrugs can be prepared by techniques known to one skilled in the art. These techniques generally modify appropriate functional groups in a given compound. These modified functional groups, however, regenerate original functional groups by routine manipulation or in vivo. Prodrugs of compounds of Formula I include compounds wherein a hydroxy, amidino, guanidino, amino, carboxylic or a similar group is modified. Examples of prodrugs include, but are not limited to, esters (e.g., acetate, formate and benzoate derivatives), carbamates (e.g., N,N-dimethylaminocarbonyl) of hydroxy functional groups in compounds of Formula I, and the like. Prodrugs of compounds of Formula I are also within the scope of this invention.

The present invention also includes N-oxide derivatives and protected derivatives of compounds of Formula I. For example, when compounds of Formula I contain an oxidizable nitrogen atom, the nitrogen atom can be converted to an N-oxide by methods well known in the art. When compounds of Formula I contain groups such as hydroxy, carboxy, thiol or any group containing a nitrogen atom(s), these groups can be protected with a suitable protecting groups. For example, Compound 2 represents a compound of Formula I containing protected hydroxy groups. A comprehensive list of suitable protective groups can be found in T. W. Greene, Protective Groups in Organic Synthesis, John Wiley & Sons, Inc. 1981, the disclosure of which is incorporated herein by reference in its entirety. The protected derivatives of compounds of Formula I can be prepared by methods well known in the art.

A “pharmaceutically acceptable salt” of a compound means a salt that is pharmaceutically acceptable and that possesses the desired pharmacological activity of the parent compound. Such salts include:
(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1′-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present invention may have asymmetric centers. Compounds of the present invention containing an asymmetrically substituted atom may be isolated in optically active or racemic forms. It is well known in the art how to prepare optically active forms, such as by resolution of materials. Many geometric isomers of olefins, C═N double bonds, and the like can be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure (representing a compound of Formula I) are intended, unless the specific stereochemistry or isomeric form is specifically indicated.

Certain compounds of Formula I exist in tautomeric equilibrium. All possible tautomers of compounds of Formula I are within the scope of this invention. Additionally, as used herein the terms alkyl includes all the possible isomeric forms of said alkyl group albeit only a few examples are set forth. Furthermore, when the cyclic groups such as aryl, heteroaryl, heterocycloalkyl are substituted, they include all the positional isomers albeit only a few examples are set forth.

“Oxo” means a radical ═O.

“Oxoalkyl” means a radical —CH₂R wherein R means a linear monovalent hydrocarbon radical of one to six carbon atoms or a branched monovalent hydrocarbon radical of three to six carbons substituted with an oxo group, as defined herein, e.g., 2-oxoethyl, 2- or 3-oxopropyl, and the like.

“Oxoheterocycloalkyl” means a saturated or unsaturated (provided that it is not aromatic) monovalent cyclic group of 3 to 8 ring atoms in which one or two ring atoms are heteroatoms selected from N, O, or S(O)n, wherein n is an integer from 0 to 2, the remaining ring atoms being C wherein one of the carbon atoms is replaced an oxo (C═O) group. The oxoheterocycloalkyl ring is optionally substituted with one or more substituents, preferably one or two substituents, independently selected from alkyl, aryl, heteroaryl, aralkyl, heteroaralkyl, haloalkyl, halo, hydroxy, cyano, carboxy, or —COOR wherein R is alkyl as define above. More specifically the term heterocycloalkyl; includes, but is not limited to, 2 or 3-oxopyrrolidin-1-yl, 2, 3, or 4-oxopiperidino, 3-oxomorpholino, 2-oxopiperazino, 2-oxotetrahydropyranyl, 3-oxothiomorpholino, 2-imidazolidone, and the substituted derivatives thereof; and the N-oxide and/or protected derivatives thereof.

“Oxoheterocycloalkylalkyl” means a radical -(alkylene)-R wherein R is a oxoheterocycloalkylalkyl group, in which oxoheterocycloalkylakyl and alkylene are as defined herein. More specifically the term oxoheterocycloalkylalkyl; includes, but is not limited to, 2 or 3-oxopyrrolidin-1-yl-(methyl, ethyl, or propyl), 2, 3, or 4-oxopiperidin-1-yl-(methyl, ethyl, or propyl), 3-oxomorpholin4-yl-(methyl, ethyl, or propyl), 2-oxopiperazin-1-yl-(methyl, ethyl, or propyl), 2-oxotetrahydropyran-3-yl-(methyl, ethyl, or propyl), 3-oxothiomorpholin-4-yl-(methyl, ethyl, or propyl), 2-imidazolidon-1-yl-(methyl, ethyl, or propyl), and the substituted derivatives thereof; and the N-oxide and/or protected derivatives thereof.

“Optional” or “optionally” means that the subsequently described event or circumstance may but need not occur, and that the description includes instances where the event or circumstance occurs and instances in which it does not. For example, “heterocycloalkyl group optionally mono- or di-substituted with an alkyl group” means that the alkyl may but need not be present, and the description includes situations where the heterocycloalkyl group is mono- or disubstituted with an alkyl group and situations where the heterocycloalkyl group is not substituted with the alkyl group.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease,
(2) inhibiting the disease, i.e., arresting or reducing the development of the disease or its clinical symptoms, or
(3) relieving the disease, i.e., causing regression of the disease or its clinical symptoms.

A “therapeutically effective amount” means the amount of a compound of Formula I that, when administered to a mammal for treating a disease, is sufficient to effect such treatment for the disease. The “therapeutically effective amount” will vary depending on the compound, the disease and its severity and the age, weight, etc., of the mammal to be treated.

“Thioureido” means a radical —NHCSNH₂.

“Ureidoalkyl” means a radical -(alkylene)-NHCONH₂ wherein alkylene is as defined herein. Representative examples include but are not limited to ureidomethyl, ureidoethyl, and the like.

“Ureido” means a radical —NHCONH₂.

“Ureidoalkyl” means a radical -(alkylene)-NHCONH₂ wherein alkylene is as defined herein. Representative examples include but are not limited to ureidomethyl, ureidoethyl, and the like.

Representative compounds of Formula I are:

TABLE I

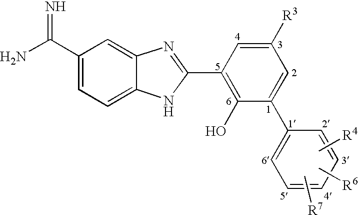

| Cpd | R³ | R⁴ | R⁶ | R⁷ |
|---|---|---|---|---|
| 1 | 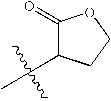 | 2′-OH | H | 5′-F |
| 2 | —CH(CH₂OC(O)CH₃)CH₂CH₂OC(O)CH₃ | 2′-OH | H | 5′-F |
| 3 | —CH(CO₂H)CH₂CH₂OH | 2′-OH | H | 5′-F |
| 4 | —CH(CH₂OH)CH₂CH₂OH | 2′-OH | H | 5′-F |
| 5 | —C(CH₂OH)₂CO₂H | 2′-OH | H | 5′-F |
| 6 | —C(CH₂OH)₂CO₂CH₃ | 2′-OH | H | 5′-CN |
| 7 | —C(CH₂OCH₃)₂CO₂H | 2′-OH | H | 5′-F |
| 8 | —C(CH₂OH)₂CO₂H | 2′-OH | H | 5′-CH₂NHCONH₂ |
| 9 | —C(CH₂OH)₂CO₂H | 2′-OH | H | 5′-CH₂NHSO₂NH₂ |
| 10 | —CH(CO₂H)CH₂C(O)H | 2′-OH | H | 5′-F |

and are named, respectively:
2-[5′-fluoro-2,2′-dihydroxy-5-(2-oxo-tetrahydro-furan-3-yl)-biphenyl-3-yl]-1H-benzoimidazole-5-carboxamidine,
acetic acid 4-acetoxy-3-[5-(5-carbamimidoyl-1H-benzoimidazol-2-yl)-5′-fluoro-6,2′-dihydroxy-biphenyl-3-yl]-butyl ester,
2-[5-(5-carbamimidoyl-1H-benzoimidazol-2-yl)-5′-fluoro-2′6-dihydroxy-biphenyl-3-yl]-4-hydroxybutanoic acid,
2-[5-(5-carbamimidoyl-1H-benzoimidazol-2-yl)-5′-fluoro-2′6-dihydroxy-biphenyl-3-yl]-4-hydroxybutan-1-ol,
2-[5-(5-carbamimidoyl-1H-benzoimidazol-2-yl)-5′-fluoro-6,2′-dihydroxybiphenyl-3-yl]-3-hydroxy-2-hydroxymethyl-propionic acid,
2-[5-(5-carbamimidoyl-1H-benzoimidazol-2-yl)-5′-cyano-6,2′-dihydroxybiphenyl-3-yl]-3-hydroxy-2-hydroxymethyl-propionic acid methyl ester,
2-[5-(5-carbamimidoyl-1H-benzoimidazol-2-yl)-5′-fluoro-6,2′-dihydroxybiphenyl-3-yl]-3-methoxy-2-methoxymethyl-propionic acid,
2-[5-(5-carbamimidoyl-1H-benzoimidazol-2-yl)-6,2′-dihydroxy-5′-ureidomethyl-biphenyl-3-yl]-3-hydroxy-2-hydroxymethyl-propionic acid,
2-[5-(5-carbamimidoyl-1H-benzoimidazol-2-yl)-6,2′-dihydroxy-5′-aminosulfonylaminomethyl-biphenyl-3-yl]-3-hydroxy-2-hydroxymethyl-propionic acid and
2-[5-(5-carbamimidoyl-1H-benzoimidazol-2-yl)-5′-fluoro-6,2′-dihydroxybiphenyl-3-yl]-4-oxo-butyric acid.

### Further Embodiments of the Invention

While the broadest definition of this invention is set forth in the Summary of the Invention, certain other definitions are intended aspects of this invention as well. For example, aspects of the invention are compounds of Formula I wherein Y, R¹ and R² each are hydrogen.

Further aspects of this invention are compounds of Formula I wherein:
X¹ is —N— and X², X³ and X⁴ are —CR⁵—, wherein R⁵ is hydrogen;
X¹ is —N—; X² and X⁴ are —CR⁵—, wherein R⁵ is hydrogen, and X³ is —CR⁵—, wherein R⁵ is halo, e.g., fluoro or chloro;
X¹ is —C=and X², X³ and X⁴ are —CR⁵—, wherein R⁵ is hydrogen; or
X¹ is —C═, X² and X⁴ are —CR⁵—, wherein R⁵ is hydrogen, and X³ is —CR⁵—, wherein R⁵ is halo, e.g., fluoro or chloro.

Aspects of this invention are compounds of Formula I wherein:
R³ is 2-hydroxy-1-hydroxymethylethyl (—CH(CH₂OH)₂), 3-hydroxy-1-hydroxymethylpropyl (—CH(CH₂OH)CH₂CH₂OH), 1-carboxy-3-hydroxypropyl (—CH(CO₂H)CH₂CH₂OH), 1-carboxy-2-methoxy-1-methoxymethylethyl (—C(CH₂OCH₃)₂CO₂H), 1-carboxy-2-hydroxy-1-hydroxymethylethyl (—C(CH₂OH)₂CO₂H) or 1-carboxy-1-hydroxymethyl-2-methoxyethyl (—C(CH₂OCH₃)(CH₂OH)CO₂H); or
R³ is 3-hydroxy-1-hydroxymethylpropyl, 1-carboxy-3-hydroxypropyl, 1-carboxy-2-methoxy-1-methoxymethylethyl, 1-carboxy-2-hydroxy-1-hydroxymethylethyl or 1-carboxy-1-hydroxymethyl-2-methoxyethyl; or
R³ is 1-carboxy-2-hydroxy-1-hydroxymethylethyl or 1-carboxy-3-hydroxypropyl.

Aspects of this invention are compounds of Formula I wherein:
R³ is a group having the formula: 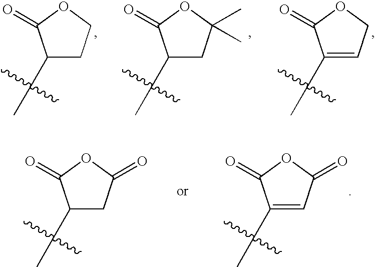
namely 2-oxo-tetrahydro-furan-3-yl, 5,5-dimethyl-2-oxo-tetrahydro-furan-3-yl, 2-oxo-2,5-dihydro-furan-3-yl, 2,5-dioxo-tetrahydro-furan-3-yl or 2,5-dioxo-2,5-dihydro-furan-3-yl, respectively; or
R³ is 2-oxo-tetrahydro-furan-3-yl.

Aspects of this invention are compounds of Formula I wherein:
R⁴ is hydroxy or hydroxymethyl and is located at the 2′-position of the biphenylyl moiety; or
R⁴ is hydroxy.

Aspects of this invention are compounds of Formula I wherein:
R⁶ is hydrogen and R⁷ is located at the 5′-position of the biphenylyl moiety and is alkyl, halo, hydroxy, hydroxyalkyl, carboxy, alkoxy, cyano, nitro, aminocarbonyl, alkylsulfonylamino, aminoalkyl, ureidoalkyl, ureido, aminosulfonylaminoalkyl, cyanoalkyl, carboxyalkyl, aminocarbonylalkyl, acyldifluoromethyl, oxoalkyl, dialkylaminocarbonylalkyl, alkoxycarbonylalkyl, cyanoalkyl or heteroaryl; or
R⁶ is hydrogen and R⁷ is located at the 5′-position of the biphenylyl moiety and is methyl, isopropyl, chloro, fluoro, hydroxymethyl, carboxy, methoxy, cyano, nitro, aminocarbonyl, methylsulfonylamino, aminomethyl, ureidomethyl, imidazol-2-yl, amino, ureido, 2-cyanoethyl, carboxymethyl, 2-carboxyethyl, aminocarbonylmethyl or dimethylaminosulfonylamino; or
R⁶ is hydrogen and R⁷ is located at the 5′-position and is hydrogen, halo, ureidomethyl or aminosulfonylaminomethyl; or
R⁶ is hydrogen and R⁷ is located at the 5′-position and is hydrogen, fluoro, ureidomethyl or aminosulfonylaminomethyl; or
R⁶ is hydrogen and R⁷ is located at the 5′-position and is fluoro or ureidomethyl; or
R is hydrogen and R⁷ is located at the 6′-position of the biphenylyl moiety and is alkyl, halo, hydroxy, hydroxyalkyl, carboxy, alkoxy, cyano, nitro, aminocarbonyl, alkylsulfonylamino, aminoalkyl, ureidoalkyl, ureido, cyanoalkyl, carboxyalkyl, aminocarbonylalkyl, acyldifluoromethyl, oxoalkyl, dialkylaminocarbonylalkyl, alkoxycarbonylalkyl, cyanoalkyl, or heteroaryl; or
R is hydrogen and R⁷ is located at the 6′-position of the biphenylyl moiety and is methyl, isopropyl, chloro, fluoro, hydroxymethyl, carboxy, methoxy, cyano, nitro, aminocarbonyl, methylsulfonylamino, aminomethyl, ureidomethyl, imidazolyl, amino, ureido, 2-cyanoethyl, carboxymethyl, 2-carboxyethyl, aminocarbonylmethyl or dimethylaminosulfonylamino; or
R⁶ is hydrogen and R⁷ is located at the 6′-position of the biphenylyl moiety and is hydroxy.

An aspect of this invention are compounds of Formula I wherein the moiety: 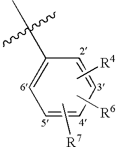
is selected from:
3′-acetylphenyl, 3′-hydroxyphenyl, 2′-hydroxyphenyl, 3′-aminocarbonylphenyl, 3′-cyanophenyl, 5′-fluoro-2′-hydroxyphenyl, 5′-chloro-2′-hydroxyphenyl, 2′-hydroxymethylphenyl, 2′-hydroxyphenyl, 5′-carboxy-2′-hydroxyphenyl, 2′,5′-dihydroxyphenyl, 5′-cyano-2′-methoxyphenyl, 5′-aminocarbonyl-2′-methoxyphenyl, 2′,6′-dihydroxyphenyl, 2′-hydroxy-5′-nitrophenyl, 2′-cyanophenyl, 3′-hydroxymethylphenyl, 5′-cyano-2′-hydroxyphenyl, 5′-aminocarbonyl-2′-hydroxyphenyl, 2′,6′-dihydroxyphenyl, 5′-aminomethyl-2′-hydroxyphenyl, 2′-hydroxy-5′-ureidomethylphenyl, 2′-hydroxy-5′-imidazol-2-ylphenyl, 5′-amino-2′-hydroxyphenyl, 2′-hydroxy-5′-ureidophenyl, 2′-hydroxy-5′-(2-morpholin-4-ylethyl)aminocarbonyl-phenyl, 3′-bromo-2′-hydroxy-5′-hydroxymethylphenyl, 5′-(2-cyanoethyl)-2′-hydroxyphenyl, 3′-bromo-5′-carboxymethyl-2′hydroxyphenyl, 5′-(2-carboxyethyl)-2′-hydroxyphenyl, 5′-aminocarbonylmethyl-2′-hydroxyphenyl, 3′,5′-dichloro-2′-hydroxyphenyl, 2′-hydroxy-5′-[2-(2-hydroxyethoxy)ethylaminocarbonyl]phenyl, 5′-dimethylaminosulfonylamino-2′-hydroxyphenyl, 3′-bromo-5′-chloro-2′-hydroxyphenyl, 2′-hydroxy-5′-(4-methylpiperazin-1-ylcarbonyl)phenyl, 2′-hydroxy-5′-(4-methylpiperazin-1-ylemthyl)phenyl, 5′-amidino-2′-hydroxyphenyl, 5′-(2-dimethylaminoethylaminocarbonyl)-2′-hydroxyphenyl and 5′-aminocarbonyl-2′-hydroxyphenyl; or
2′-hydroxyphenyl, 5′-fluoro-2′-hydroxyphenyl, 5′-chloro-2′-hydroxyphenyl, 2′-hydroxymethylphenyl, 2′-hydroxyphenyl, 5′-carboxy-2′-hydroxyphenyl, 2′,5′-dihydroxyphenyl, 2′,6′-dihydroxyphenyl, 2′-hydroxy-5′-nitrophenyl, 5′-cyano-2′-hydroxyphenyl, 5′-aminocarbonyl-2′-hydroxyphenyl, 2′,6′-dihydroxyphenyl, 5′-aminomethyl-2′-hydroxyphenyl, 2′-hydroxy-5′-ureidomethylphenyl, 2′-hydroxy-5′-imidazol-2-ylphenyl, 5′-amino-2′-hydroxyphenyl, 2′-hydroxy-5′-ureidophenyl, 2′-hydroxy-5′-(2-morpholin-4-ylethyl)aminocarbonyl-phenyl, 3′-bromo-2′-hydroxy-5′-hydroxymethylphenyl, 5′-(2-cyanoethyl)-2′-hydroxyphenyl, 3′-bromo-5′-carboxymethyl-2′-hydroxyphenyl, 5′-(2-carboxyethyl)-2′-hydroxyphenyl, 5′-aminocarbonylmethyl-2′-hydroxyphenyl, 3′,5′-dichloro-2′-hydroxyphenyl, 2′-hydroxy-5′-[2-(2-hydroxyethoxy)ethylaminocarbonyl]phenyl, 5′-dimethylaminosulfonylamino-2′-hydroxyphenyl, 3′-bromo-5′-chloro-2′-hydroxyphenyl, 2′-hydroxy-5′-(4-methylpiperazin-1-ylcarbonyl)phenyl, 2′-hydroxy-5′-(4-methylpiperazin-1-ylmethyl)phenyl, 5′-carbamimidoyl-2′-hydroxyphenyl, 5′-(2-dimethylaminoethylaminocarbonyl)-2′-hydroxyphenyl, 3-nitrophenyl and 5′-aminocarbonyl-2′-hydroxyphenyl; or
2′,6′-dihydroxyphenyl, 5′-fluoro-2′-hydroxy-phenyl, 5′-aminocarbonyl-2′-hydroxyphenyl and 2′-hydroxy-5′-ureidomethylphenyl.

An aspect of this invention are compounds of Formula I wherein:
Y, R¹ and R² each are hydrogen;
X¹ is —N— and X², X³, and X⁴ are —CR⁵—, wherein R⁵ is hydrogen;
R³ is 2-hydroxy-1-hydroxymethylethyl, 3-hydroxy-1-hydroxymethylpropyl, 1-carboxy-3-hydroxypropyl, 1-carboxy-2-methoxy-1-methoxymethylethyl, 1-carboxy-2-hydroxy-1-hydroxymethylethyl, 1-carboxy-1-hydroxymethyl-2-methoxyethyl, 2-oxo-tetrahydro-furan-3-yl, 5,5-dimethyl-2-oxo-tetrahydro-furan-3-yl, 2-oxo-2,5-dihydro-furan-3-yl, 2,5-dioxo-tetrahydro-furan-3-yl or 2,5-dioxo-2,5-dihydro-furan-3-yl;
R⁴ is hydroxy or hydroxymethyl and is located at the 2′-position of the biphenylyl moiety; and
R⁶ is hydrogen and R⁷ is located at the 5′-position of the biphenylyl moiety and is alkyl, halo, hydroxy, hydroxyalkyl, carboxy, alkoxy, cyano, nitro, aminocarbonyl, alkylsulfonylamino, aminoalkyl, ureidoalkyl, ureido, aminosulfonylaminoalkyl, cyanoalkyl, carboxyalkyl, aminocarbonylalkyl, heteroaryl.

An aspect of this invention are compounds of Formula I wherein:
Y, R¹ and R² each are hydrogen;
X¹ is —N— and X², X³, and X⁴ are —CR⁵—, wherein R⁵ is hydrogen;
R³ is 3-hydroxy-1-hydroxymethylpropyl, 1-carboxy-3-hydroxypropyl, 1-carboxy-2-methoxy-1-methoxymethylethyl, 1-carboxy-2-hydroxy-1-hydroxymethylethyl, 1-carboxy-1-hydroxymethyl-2-methoxyethyl or 2-oxo-tetrahydro-furan-3-yl;
R⁴ is hydroxy; and
R⁶ is hydrogen and R⁷ is located at the 5′-position and is methyl, isopropyl, chloro, fluoro, hydroxymethyl, carboxy, methoxy, cyano, nitro, aminocarbonyl, methylsulfonylamino, aminomethyl, ureidomethyl, imidazol-2-yl, amino, ureido, 2-cyanoethyl, carboxymethyl, 2-carboxyethyl, aminocarbonylmethyl, dimethylaminosulfonylamino.

An aspect of this invention are compounds of Formula I wherein:
Y, R¹ and R² each are hydrogen;
X¹ is —N— and X², X³, and X⁴ are —CR⁵—, wherein R⁵ is hydrogen;
R³ is 3-hydroxy-1-hydroxymethylpropyl, 1-carboxy-3-hydroxypropyl, 1-carboxy-2-methoxy-1-methoxymethylethyl, 1-carboxy-2-hydroxy-1-hydroxymethylethyl, 1-carboxy-1-hydroxymethyl-2-methoxyethyl or 2-oxo-tetrahydro-furan-3-yl;
R⁴ is hydroxy; and
R⁶ is hydrogen and R⁷ is located at the 5′-position and is hydrogen, halo, ureidomethyl or aminosulfonylaminomethyl.

An aspect of this invention are compounds of Formula I wherein:
Y, R¹ and R² each are hydrogen;
X¹ is —N— and X², X³, and X⁴ are —CR⁵—, wherein R⁵ is hydrogen;
R³ is 1-carboxy-2-hydroxy-1-hydroxymethylethyl or 1-carboxy-3-hydroxypropyl;
R⁴ is hydroxy; and
R⁶ is hydrogen and R⁷ is located at the 5′-position and is fluoro or ureidomethyl.

### General Synthetic Scheme

Compounds of this invention can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compounds of Formula I in which Y is hydrogen and X¹ is —N—, i.e., a compound of Formula I(a), can be prepared as described in Scheme I: 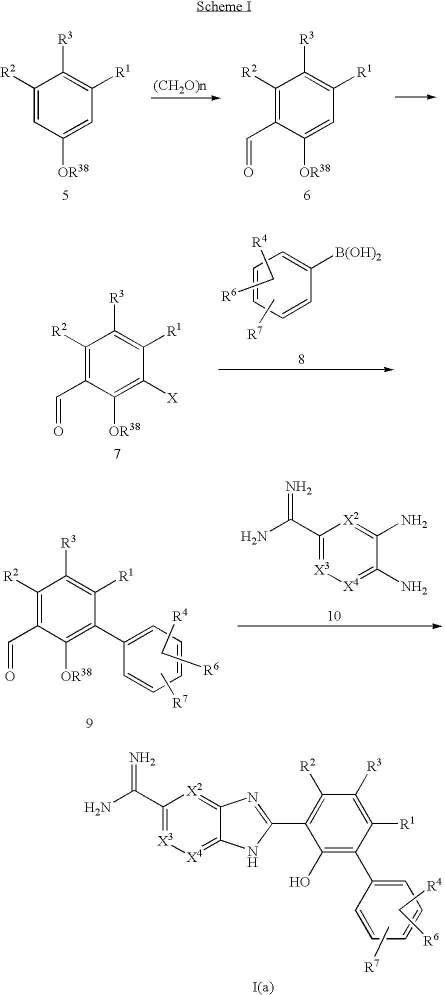
wherein X is halo, preferably bromo or iodo, R³⁸ is hydrogen or a suitable protecting group and each X², X³, X⁴, R¹, R², R³, R⁴, R⁶ and R⁷ are as defined in the Summary of the Invention.

Compounds of Formula I(a) can be prepared by condensing a compound of Formula 9 with a diamine of Formula 10 and then removing any protecting groups present. The condensation reaction typically is carried out in the presence of a suitable oxidizing agent (e.g., 1,4-benzoquinone and the like) or with air oxidation and in a suitable solvent (e.g., methanol, ethanol, DMF and the like) at 60 to 120° C. and requires 0.5 to 12 hours to complete. Detailed descriptions for the preparation of a compound of Formula I(a) by the methods described above are set forth in Examples 1 and 4, infra.

Methods for protecting reactive groups and removing protecting groups present in a compound are well known in the art. A comprehensive list of suitable protective groups and procedures for their addition and removal can be found in T. W. Greene, Protective Groups in Organic Synthesis, John Wiley & Sons, Inc. 1981, the disclosure of which is incorporated herein by reference in its entirety. For example, compounds with protected hydroxy groups can be prepared by reacting the corresponding unprotected compound with 2-methoxyethoxymethyl chloride (MEM-Cl) to give the corresponding MEM protected compound. The reaction typically is carried out the presence of a suitable base (e.g., N,N-diisopropylethylamine and the like) and in a suitable solvent (e.g., dichloromethane or the like) at 0 to 25° C. and requires 1 to 12 hours to complete. Detailed descriptions for the preparation of a MEM protected compound by the methods described above are set forth in References 2 and 9, infra. A detailed description for the preparation of a acetyl protected compound is set forth in Reference 11, infra. A detailed description for deprotecting acetyl protected hydroxy groups is set forth in Example 3, infra. A person skilled in the art will recognize that other suitable hydroxy protecting groups can be used as well.

Compounds of Formula 10 are commercially available (e.g., 3,4-diaminobenzamidine, 3,4-diamino-6-fluoro-benzamidine, and the like) or can be prepared by methods well known in the art.

Compounds of Formula 9 can be prepared by reacting a compound of Formula 7 with a boronic acid of Formula 8. The reaction typically is carried out in the presence of a suitable coupling agent (e.g., tetrakis-(triphenylphosphine)palladium) in a suitable solvent (e.g., toluene,1,2-dimethoxy-ethane, and the like) at 80 to 120° C. and requires 1 to 12 hours to complete. Alternatively the boronic acid of Formula 8 can be substituted by a corresponding 4,4,5,5-tetramethyl-2-phenyl-[1,3,2]dioxaborolane derivative. Detailed descriptions for the preparation of a compound of Formula 9 by the methods described above are set forth in References 7 and 13, infra.

Compounds of Formula 8 can be prepared by reacting a corresponding substituted bromobenzene derivative with trimethylborate. The reaction with the with trimethylborate typically is carried out in the presence of a metallorganic reagent (e.g., n-butyllithium, Grignard reagents, and the like) and in a suitable solvent (e.g., tetrahydrofuran (THF), ethyl ether, and the like) at −78 to 0° C. and requires 30 to 320 minutes to complete. The 4,4,5,5-tetramethyl-2-phenyl-[1,3,2]dioxaborolanes can be prepared by reacting a corresponding substituted bromobenzene derivative with bis(pinacolato)diboron. The reaction with the bis(pinacolato)diboron typically is carried out in the presence of a suitable catalyst (e.g., dichloro[1,1′bis(diphenylphosphino)ferrocene]-palladium(II)-dichloromethane adduct or the like) and in a suitable solvent (e.g., dioxane or the like) at 80 to 100° C. and requires 30 to 120 minutes to complete. Detailed descriptions for the preparation of a compound of Formula 9 and a 4,4,5,5-tetramethyl-2-phenyl-[1,3,2]dioxaborolane by the methods described above are set forth in References 1 and 2, infra.

Compounds of Formula 7 can be prepared by reacting a corresponding phenol, e.g., a compound of Formula 5(a), with paraformaldehyde to give the corresponding aldehyde of Formula 6 and then halogenating the aldehyde to give the corresponding halo-formyl-substituted compound of Formula 7. The reaction with parformaldehyde typically is carried out in the presence of magnesium chloride and a suitable base (e.g., triethylamine or the like) and in a suitable solvent (e.g., acetonitrile, tetrahydrofuran, dioxane and the like) at 60 to 100° C. and requires 30 to 320 minutes to complete. The halogenation may be effected with a suitable halogenating agent (e.g., N-bromo succinimide, N-iodosuccinimide, and the like) in a suitable solvent (e.g., N,N-dimethylformamide (DMF) or the like) at 0 to 25° C. and requires 60 to 120 minutes to complete. A detailed description for the preparation of a compound of Formula 7 by the methods described above is set forth in Reference 4, infra.

Compounds of Formula 5 are commercially available or can be prepared by methods well known in the art. For example, a compound of Formula 5 in which R³ is a group of formula (a) wherein n is 1, i.e., a compound of Formula 5(a), can be prepared as described in Scheme II: 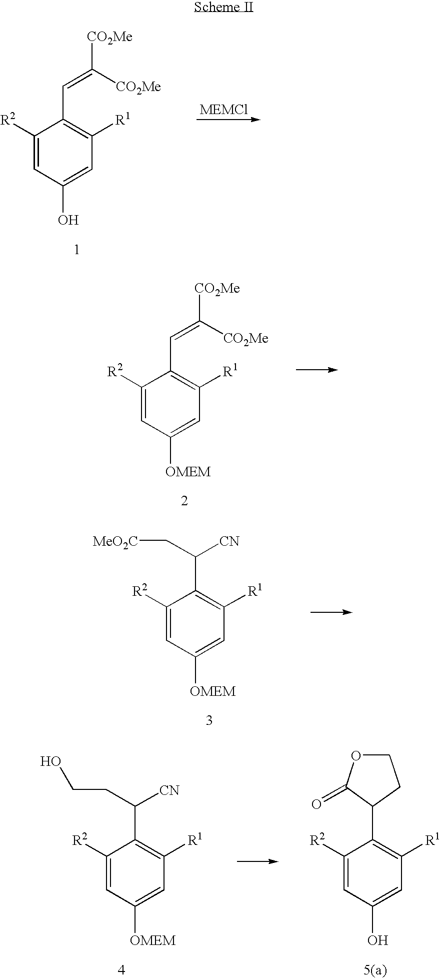
in which MEMO is 2-methoxyethoxymethoxy and each R¹ and R² are as defined in the Summary of the Invention.

A compound of Formula 5(a) can be prepared from the corresponding compound of Formula 1. The compound of Formula 1 is protected to give a compound of Formula 2 which then is converted to the compound of Formula 5 by the procedures described in Gu, J. X and Holland, H. L., Synth. Commun. Vol. 28, No. 18, p. 3305-3315 (1998). A detailed description for the preparation of a compound of Formula 5(a) by the methods described above is set forth in Reference 10, infra. A detailed description for the preparation of a compound of Formula 2 is set forth in Reference 9, infra.

Compounds of Formula 5 in which R³ is a group of formula (c), i.e., a compound of Formula 5(b), can be prepared as described in Scheme III: 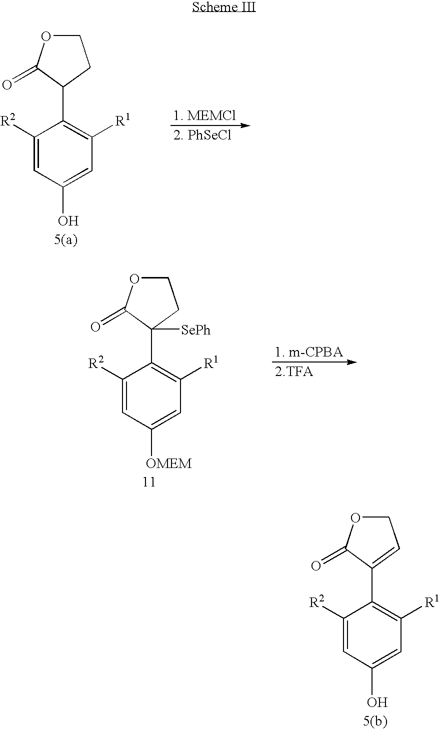
in which MEMO is 2-methoxyethoxymethoxy and each R¹ and R² are as defined in the Summary of the Invention.

Compounds of Formula 5(b) can be prepared from the corresponding compound of Formula 5(a). The compound of Formula 5(a) is treated with MEM-CI to provide the MEM protected derivative, which is then treated with phenylselenenyl chloride to provide a compound of Formula 11. The compound of Formula 11 is then treated with m-chloroperoxobenzoic acid to provide the dehydrogenated derivative, which is then deprotected to provide the compound of Formula 5(b). The reaction with the MEM-Cl is carried out as provided above. The reaction with the phenylselenenyl chloride and subsequent dehydrogenation is carried out under conditions described in Gu, J. X and Holland, H. L., Synth. Commun. Vol. 28, No. 18, p. 3305-3315 (1998).

Compounds of Formula 5 in which R³ is a group of formula (b) wherein n is 1, i.e., a compound of Formula 5(c), can be prepared as described in Scheme IV: 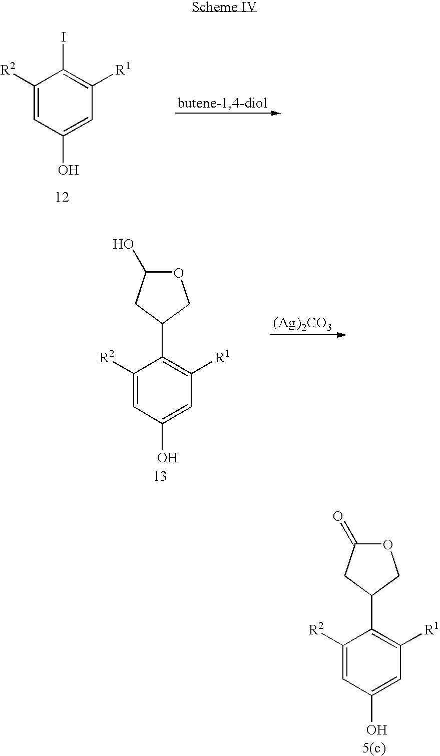
in which each R¹ and R² are as defined in the Summary of the Invention.

Compounds of Formula 5(c) can be prepared by reacting a compound of Formula 12 with butene-1,4-diol to provide a compound of Formula 13 and then oxidizing the compound of Formula 13. The reaction with the diol can be carried out in the presence of a suitable catalyst (e.g., palladium(II)acetate or the like) with tetrabutylammonium chloride and potassium carbonate in a suitable solvent (e.g., DMF or the like) at about 60° C. The oxidation is carried with an oxidizing agent (e.g., Fetisons reagent or the like) in a suitable solvent (e.g., toluene and the like) at about 80° C. under conditions described in Arcadi A. et al. Tetrahedron, v. 47, N8, 1991, p. 1525-40.

Compounds of Formula 5 in which R³ is cyclopropyl substituted with carboxy or carboxyalkyl, i.e., compounds of Formulae 5(d) and 5(e), respectively, can be prepared as described in Scheme IV: 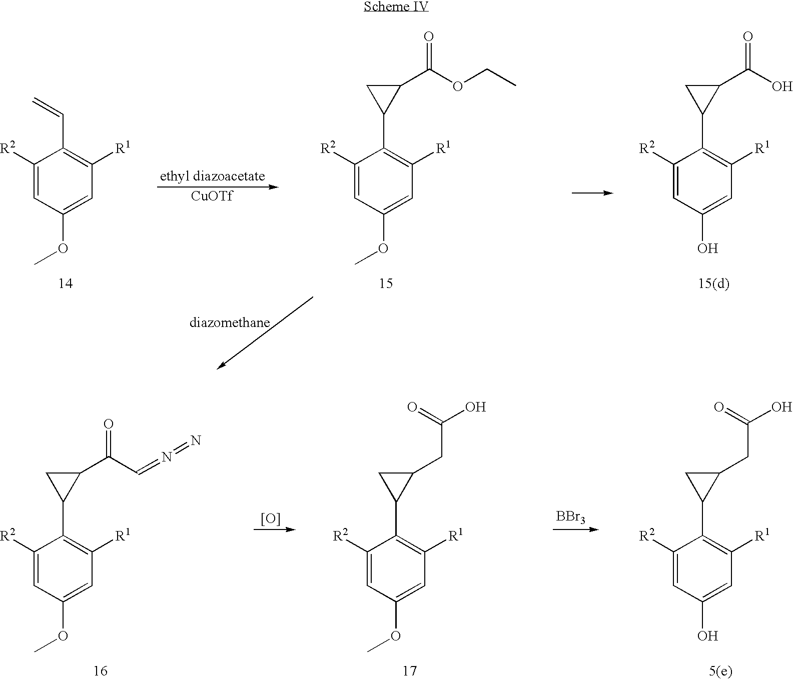
in which each R¹ and R² are as defined in the Summary of the Invention.

Compounds of Formula 5(d) can be prepared by reacting a compound of Formula 14 with ethyl diazoacetate and then deprotecting. The reaction with the acetate can be carried in the presence of a catalytic amount of copper(II) trifluoromethanesulfonate under conditions described in J. Chem. Soc. Dalton Trans., Vol. 6, p 1043-1046 (1998).

Compounds of Formula 5(e) can be prepared by reacting a compound of Formula 15 with diazomethane to provide a compound of Formula 16, oxidizing to provide a compound of Formula 17 and then deprotecting. The reaction with the diazomethane is carried by treating the compound of Formula 15 with LiOH, then activating with thionyl chloride and reacting the activated intermediate with the diazomethane in a suitable solvent (e.g., DMF or the like). The oxidation is carried out with a suitable oxidizing agent (e.g., silver(I) oxide or the like) and aqueous sodium thiosulfate in a suitable solvent (e.g., dioxane or the like). Deprotection can be effected with boron tribromide.

Compounds of Formula 5 in which R³ is cyclopentyl or cyclohexyl substituted with protected carboxymethyl, i.e., a compounds of Formulae 5(f), can be prepared as described in Scheme V: 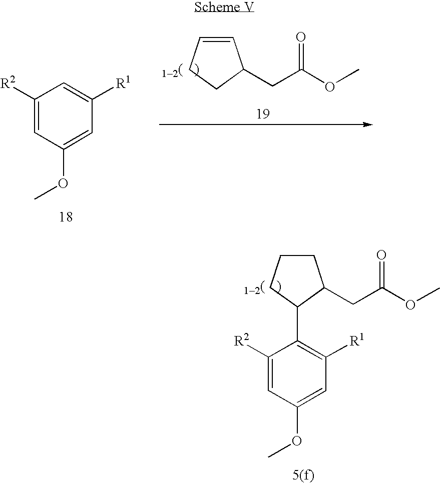
in which each R¹ and R² are as defined in the Summary of the Invention.

Compounds of Formula 5(f) can be prepared by condensing a compound of Formula 18 with a compound of Formula 19 in the presence of aluminum chloride. A detailed description of the reaction for carrying out the condensation reaction is set forth in Bull. Soc. Chim. Fr., 1035, 1040 (1952).

Compounds of Formula 5 in which R³ is cyclopentyl or cyclohexyl substituted with protected carboxymethyl, i.e., a compounds of Formulae 5(g), can be prepared as described in Scheme VI: 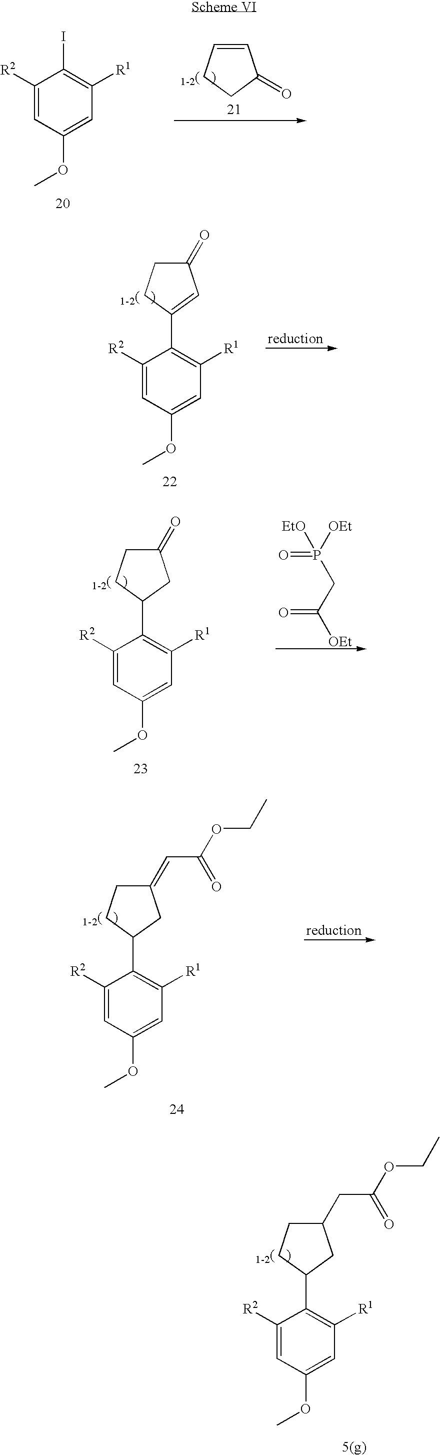
in which each R¹ and R² are as defined in the Summary of the Invention.

Compounds of Formula 5(g) can be prepared by reacting a compound of Formula 23 with ethyl diethylphosphorylacetate to provide a compound of Formula 24 and then reducing. Compounds of Formula 23 can be prepared by condensing a compound of Formula 20 with a compound of Formula 21 to provide a compound of Formula 22 and then reducing the compound of Formula 22. A detailed description of conditions for effecting the reactions in Scheme VI are set forth in Collect. Czech. Chem. Commun, 51, 12, p 2896-2908 (1986).

Compounds of Formula 5 in which R³ is protected carboxyalkyl substituted with tetrazol-5-yl, i.e., a compounds of Formulae 5(h), can be prepared as described in Scheme VII: 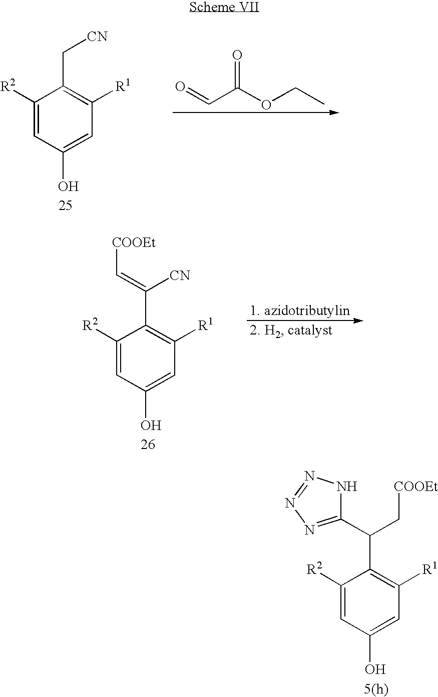
in which each R¹ and R² are as defined in the Summary of the Invention.

Compounds of Formula 5(h) can be prepared by condensing a compound of Formula 25 with ethyl 2-oxoacetate to provide a compound of Formula 26 and then reacting the compound of Formula 26 with azidotributyltin. The condensation reaction is carried out in the presence of base under conditions well known in the art. The reaction with the azidotributylin is followed by reduction of the double bond with hydrogenation in the presence of a suitable reducing catalyst under conditions well known in the art.

Compounds of Formula 5 in which R³ is protected dicarboxyalkyloxy, i.e., a compounds of Formula 5(i), can be prepared as described in Scheme VIII: 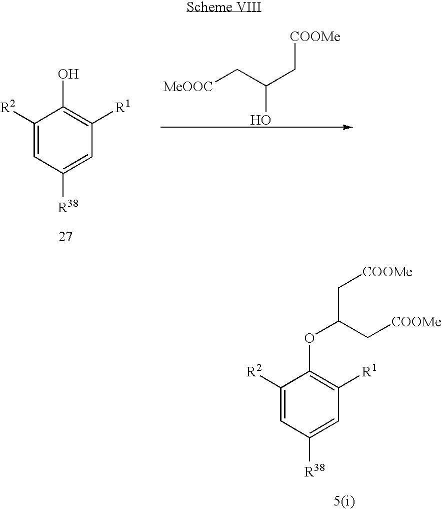
in which R³⁸ is hydrogen or a suitable protecting group and each R¹ and R² are as defined in the Summary of the Invention.

Compounds of Formula 5(i) can be prepared by reacting a compound of Formula 27 with methyl 3-hydroxy-4-methoxycarbonylbutanoate. The reaction can be carried out in the presence of diethyl azodicarboxylate and triphenylphosphine in tetrahydrofuran at about 0° C. and requires about 2 hours for completion.

Compounds of Formula 5 in which R³ is protected 3-hydroxy-1-hydroxymethylpropyl, i.e., compounds of Formula 5(b), can be prepared as described in Scheme IX: 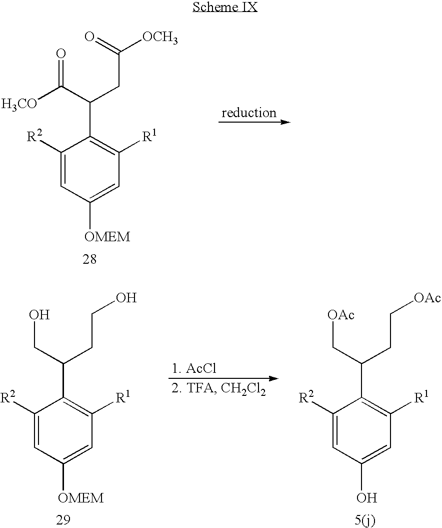
in which MEMO is 2-methoxyethoxymethoxy and each R¹ and R² are as defined in the Summary of the Invention.

Compounds of Formula 5(j) can be prepared by acylating a compound of Formula 29. The acylation is carried out with a suitable acylating agent (e.g, acetyl chloride, acetic anhydride, and the like) in the presence of a base (e.g., pyridine, triethylamine and the like) and in a suitable solvent (e.g., dichloromethane, benzene, tetrahydrofuran and the like) at 0 to 25° C. and requires 30 to 120 minutes to complete. A detailed description for the preparation of a compound of Formula 5(j) is set forth in Reference 11, infra.

Compounds of Formula 29 can be prepared by reducing a corresponding compound of Formula 28. The reduction is carried out with a suitable reducing agent (e.g., lithium aluminum hydride, diisobutylaluminum hydride (DIBAL), and the like) in a suitable etheral solvent (e.g., tetrahydrofuran, diethyl ether, and the like) at 0 to 40° C. and requires 30 to 120 minutes to complete. A detailed description for the preparation of a compound of Formula 28 is set forth in Reference 11, infra.

Compounds of Formula I in which Y is hydrogen, X¹ is —N— and R³ is 3-hydroxy-1-hydroxymethylpropyl, i.e., compounds of Formula I(b), can be prepared as described in Scheme IX: 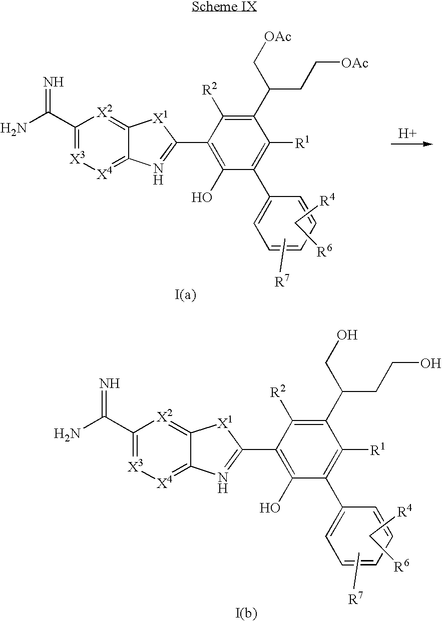
in which each X¹, X², X⁴, R¹, R², R³, R⁴, R⁶, and R⁷ are as defined in the Summary of the Invention.

Compounds of Formula I(b) can be prepared by hydrolysis of the corresponding protected compound of Formula I(a). The hydrolysis reaction is carried out in the presence of a suitable acid (e.g., aqueous hydrochloric acid or the like) and in a suitable solvent (e.g., acetonitrile, acetic acid, and the like) at 80 to 120° C. and requires 60 to 720 minutes to complete. A detailed description for the preparation of a compound of Formula I(b) is set forth in Example 4, infra.

Compounds of Formula I(a) can be prepared from the corresponding compounds of Formulae 9 and 10 by the methods set for in Scheme I for the preparation of compounds of Formula I and further described by the methods set forth in Example 1, infra.

Compounds of Formula I in which R³ is 3,5-dioxo-cyclopent-1-enyl, i.e. a compound of Formula I(c), can be prepared as described in Scheme X: 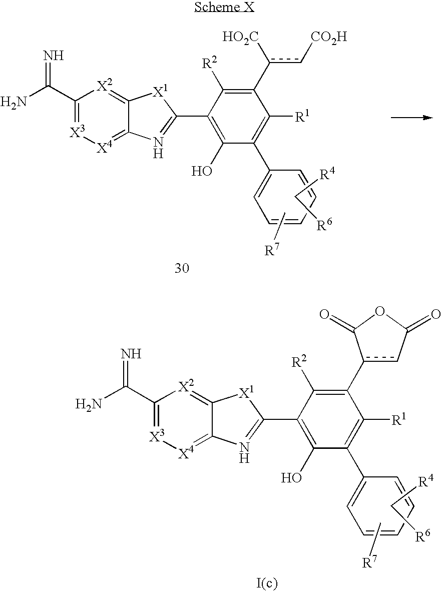
in which the dashed line represents an optional bond and each X¹, X², X³, X⁴, R¹, R², R³, R⁴, R⁶, and R⁷ are as defined in the Summary of the Invention.

Compounds of Formula I(c) can be prepared by treating a compound of Formula 30 with a coupling agent (e.g., 1-(3-dimethylaminopropyl)-3-ethylcarbodiimide hydrochloride (EDC) or the like) and a suitable base (e.g., triethylamine or the like) in a suitable solvent (DMF or the like). The reaction is carried out at about 0° C. to ambient temperature and requires 1 to 10 hours for completion.

Compounds of Formula I wherein X¹ is —CH— can be prepared utilizing the procedures described in U.S. Provisional Patent Application Ser. No. 60/190,147 and PCT International Patent Application WO 00/35886 and Applicants' U.S. Provisional Application titled “2-(2-Hydroxybiphenyl-3-yl)-1H-benzoimidazole-5-carboxamidine derivatives as factor VIIa inhibitors”, filed on Dec. 3, 2002, the disclosures of which are incorporated herein by reference in their entirety.

Compounds of Formula I wherein R³ is 1-carboxy-3-hydroxypropyl can be prepared by treating a compound of Formula I wherein R³ is tetrahydrafuran-2-one with a suitable base (e.g., aqueous sodium hydroxide, alcoholic potassium hydroxide, and the like) at 0 to 25° C. for 30 to 120 minutes. Suitable alcohol solvents include methanol, ethanol, and the like. A detailed description for the preparation of a compound of Formula I wherein R³ is 1-carboxy-3-hydroxypropyl is set forth in Example 2, infra.

### Utility

The compounds of this invention are inhibitors of Factors VIIa and Xa, in particular Factor VIIa, and are therefore useful as anticoagulants for the treatment or prevention of thromboembolic disorders in mammals. The term “thromboembolic disorders” as used herein includes arterial or venous cardiovascular or cerebrovascular thromboembolic disorders, including, for example unstable angina, first or recurrent ischemic attack, stroke, atherosclerosis, venous thrombosis, deep vein thrombosis, thrombophlebitis, arterial embolism, kidney embolisms, and pulmonary embolisms.

### Testing

The ability of the compounds of this invention to inhibit factor VIIa or IXa can be tested in vitro and in vivo assays described in biological assays Example 1 and 2 below.

### Administration and Pharmaceutical Compositions

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors.

Therapeutically effective amounts of compounds of Formula I may range from approximately 0.1-50 mg per kilogram body weight of the recipient per day; preferably about 0.5-20 mg/kg/day. Thus, for administration to a 70 kg person, the dosage range would most preferably be about 35 mg to 1.4 g per day.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral or parenteral using a convenient daily dosage regimen, which can be adjusted according to the degree of affliction. Oral compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of Formula I in combination with at least one pharmaceutically acceptable excipient. Acceptable excipients are non-toxic, aid administration, and do not adversely affect the therapeutic benefit of the compound of Formula I. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of Formula I based on the total formulation, with the balance being one or more suitable pharmaceutical excipients. Preferably, the compound is present at a level of about 1-80 wt %. Representative pharmaceutical formulations containing a compound of Formula I are described below.

### EXAMPLES

The following preparations and examples are given to enable those skilled in the art to more clearly understand and to practice the present invention. They should not be considered as limiting the scope of the invention, but merely as being illustrative and representative thereof.

### Synthetic Examples

### Reference 1

### Synthesis of (5-Fluoro-2-methoxymethoxy-phenyl)-boronic acid

Commercially available 2-bromo-4-fluorophenol (25.0 g, 0.13 mol) was dissolved in dry dichloromethane (100 mL) and dimethoxymethane (115 mL, 1.30 mol). Phosphorus pentoxided (110.8 g, 0.39 mol) was added portion-wise, while keeping the reaction temperature below 40° C. The mixture was stirred vigorously at room temperature for two hours then carefully poured into 1N aqueous sodium hydroxide (50 mL). The organic layer was collected, washed with water and brine, dried over anhydrous MgSO₄, filtered and concentrated to give 2-bromo-4-fluoro-1-methoxymethoxy-benzene as a colorless oil (30.1 g, 100%). HPLC (C-18 reverse phase) 8.280 min (1-1-90).

A 500 mL round bottom flask was charged with a 1.6M solution of n-butyllithium in hexanes (100 mL, 0.16 mol), flushed with nitrogen and the solution was cooled to −78° C. 2-bromo-4-fluoro-1-methoxymethoxy-benzene (30.1 g, 0.13 mol) was dissolved in 50 mL of dry tetrahydrofuran (THF) and the solution was added dropwise to the cooled n-butyllithium solution over one hour. The mixture was stirred for one hour at −78° C. and then trimethylborate (20 mL, 0.175 mol) was added very slowly via syringe. The reaction was allowed to gradually warm to room temperature and after two hours the mixture was poured into ice, acidified to pH 4 with 5% aqueous citric acid and extracted with ethyl acetate. The combined organic extracts were washed with water and brine, dried over MgSO₄ and filtered. Evaporation of the solvent under reduced pressure gave crude material (28.2 g). Recrystallization from hexane gave (5-fluoro-2-methoxymethoxy-phenyl)-acetic acid boronic acid (18.9 g, 73%). ¹H NMR (CDCl₃) δ ppm: 7.51 (d, J=2.1 Hz, 1H), 7.08 (m, 2H), 5.92 (bs, 2H), 5.25 (s, 2H), 3.51 (s, 3H).

### Reference 2

### Synthesis of 4-(2-methoxy-ethoxymethoxy)-3-(4,4,5,5-tetramethyl-[1,3,2]dioxaborolan-2-yl)-benzonitrile

MEM-Cl (7.87 g, 7.21 mL, 63.2 mmol) and N,N-diisopropylethylamine (8.84 g, 11.9 mL, 68.4 mmol) were added to 3-bromo-4-hydroxy-benzonitrile (10.0 g, 52.6 mmol) in solution with dichloromethane (250 mL). The mixture was stirred 2 hours and the organic layer was washed with water, dried (MgSO₄) and concentrated to give 3-bromo-4-(2-methoxy-ethoxymethoxy)-benzonitrile. HPLC (C-18 reverse phase) 3.734 min (1-90S).

3-Bromo-4-(2-methoxy-ethoxymethoxy)-benzonitrile (5.20 g, 18.2 mmol), dioxane (180 mL), bis(pinacolato)diboron (5.54 g, 21.8 mmol) and potassium acetate (5.35 g, 54.5 mmol) were combined in a 250 mL 24/40 round bottom flask. Argon was bubbled through the mixture and then dichloro[1,1′bis(diphenylphosphino)ferrocene]-palladium(II)-dichloromethane adduct (purchased from STREM cat# 46-0450) (0.74 g, 0.91 mmol) was added, The solution was refluxed for 4 hours, cooled and then diluted with ethyl acetate (100 mL). The mixture was washed with 5% citric acid and then brine and dried. The solvent is removed under reduced pressure. The residue was taken up in toluene (180 mL) to generate a 0.1 M solution of 4-(2-methoxy-ethoxymethoxy)-3-(4,4,5,5-tetramethyl-[1,3,2]dioxaborolan-2-yl)-benzonitrile.

### Reference 3

### Synthesis of 2-(4-hydroxy-phenyl)-3-methoxy-2-methoxymethyl-propionic acid methyl ester

3-Methoxy-2-[4-(2-methoxy-ethoxymethoxy)-phenyl]-2-methoxymethyl-propionic acid methyl ester (0.34 g, 1 mmol) was dissolved in methanol (5 mL) and 4N hydrogen chloride in dioxane (5 ml) was added to the solution. The mixture was stirred for 4 hours and then the solvents were removed in vacuum. The residue was partitioned between water and ethyl acetate and the organic layer was separated and washed with water and brine and dried over magnesium sulfate. The solvent was then removed in vacuum to provide 2-(4-hydroxyphenyl)-3-methoxy-2-methoxymethyl-propionic acid methyl ester (0.22 g, 86%).

### Reference 4

### Synthesis of 2-(3-bromo-5-formyl-4-hydroxy-phenyl)-3-methoxy-2-methoxymethyl-propionic acid methyl ester

Magnesium chloride (0.22 g, 2.25 mmol) was added in one portion to stirring mixture of 2-(4-hydroxy-phenyl)-3-methoxy-2-methoxymethyl-propionic acid methyl ester (0.22 g, 0.86 mmol), paraformaldehyde (0.32 g, 10.5 mmol) and triethylamine (0.75 g, 7.5 mmol) in acetonitrile (15 ml). The mixture was heated under reflux for 4 hours, cooled and then poured into cold 1N aqueous hydrochloric acid. The product was extracted with ethyl acetate and the extract was washed with water and then brine and dried over magnesium sulfate. The solvent was removed in vacuum to give crude 2-(3-formyl-4-hydroxy-phenyl)-3-methoxy-2-methoxymethyl-propionic acid methyl ester, which was purified by column chromatography on silicagel (eluent—hexane-ethyl acetate, 7:3), yielding 0.063 g, 26%).

A solution of N-bromosuccinimide (0.05 g, 0.28 mmol) in DMF (3 ml) was added dropwise to 2-(3-formyl-4-hydroxy-phenyl)-3-methoxy-2-methoxymethyl-propionic acid methyl ester (0.063 g, 0.22 mmol) in a solution with DMF (3 ml). The mixture was stirred for 3 hours and then partitioned between water and ethyl acetate. The organic layer was separated and washed with water and then brine and dried over magnesium sulfate. The solvent was removed to give 2-(3-bromo-5-formyl-4-hydroxy-phenyl)-3-methoxy-2-methoxymethyl-propionic acid methyl ester (0.077 g, 97%).

### Reference 5

### Synthesis of 2-(4-benzyloxy-3-bromo-5-formyl-phenyl)-3-hydroxy-2-hydroxymethyl-propionic acid methyl ester

A mixture of (4-benzyloxy-3-bromo-5-formyl-phenyl)-acetic acid methyl ester (0.36 g, 1 mmol), paraformaldehyde (0.3 g, 10 mmol) and potassium tert-butoxide (0.11 g, 1 mmol) in DMF (10 mL) was stirred for 16 hours and then poured into 1N aqueous hydrochloric acid. The mixture was extracted with ethyl acetate and the organic layer was washed with water and brine, dried over magnesium sulfate and concentrated. The residue was subjected to flash chromatography on silica using hexane-ethyl acetate (8:2) mixture for elution to give 2-(4-benzyloxy-3-bromo-5-formyl-phenyl)-3-hydroxy-2-hydroxymethyl-propionic acid methyl ester (0.11 g, 26%).

Proceeding as in Reference 5, but substituting [5′-cyano-5-formyl-6,2′-bis-(2-methoxy-ethoxymethoxy)-biphenyl-3-yl]-acetic acid methyl ester gave 2-[5′-cyano-5-formyl-6,2′-bis-(2-methoxy-ethoxymethoxy)-biphenyl-3-yl]-3-hydroxy-2-hydroxymethyl-propionic acid methyl ester (45%).

Proceeding as in Reference 5, but substituting [4-(2-methoxy-ethoxymethoxy)-phenyl]-acetic acid methyl ester gave 3-hydroxy-2-hydroxymethyl-2-[4-(2-methoxyethoxymethoxy)-phenyl]-propionic acid methyl ester (33%).

### Reference 6

### Synthesis of 3-methoxy-2-[4-(2-methoxy-ethoxymethoxy)-phenyl]-2-methoxymethyl-propionic acid methyl ester

3-Hydroxy-2-hydroxymethyl-2-[4-(2-methoxy-ethoxymethoxy)-phenyl]-propionic acid methyl ester (0.6 g, 1.9 mmol), prepared as in Reference 5, in a solution was added dropwise to a suspension of sodium hydride (0.14 g, 5.8 mmol) in DMF (7 ml). The mixture was stirred for 30 minutes and then cooled to 10° C. Methyl iodide (0.85 g, 5.7 mmol) was added in small portions and the mixture was stirred for 14 hours, quenched with saturated aqueous ammonium chloride and extracted with ethyl acetate. The organic layer was washed with water, brine, dried (magnesium sulfate) and concentrated in vacuum to give (0.49 g, 75%).

### Reference 7

### Synthesis of 2-(5′-fluoro-5-formyl-6-hydroxy-2′-methoxymethoxy-biphenyl-3-yl)-3-methoxy-2-methoxymethyl-propionic acid methyl ester

2-(3-Bromo-5-formyl-4-hydroxy-phenyl)-3-methoxy-2-methoxymethyl-propionic acid methyl ester (0.077 g, 0.21 mmol), prepared as in Reference 4, and 5-fluoro-2-methoxymethoxy-phenyl-boronic acid (0.052 g, 0.26 mmol), prepared as in Reference 1, were dissolved in dimethoxyethane (5 ml) and then aqueous sodium carbonate (2M, 0.11 ml, 0.22 mmol) and tetrakis(triphenylphoshine)palladium (0.013 g, 0.011 mmol) were added to the solution. The mixture was heated under reflux for 4 hours, cooled and mixed with 1 N aqueous hydrochloric acid. The crude product was extracted with ethyl acetate and the organic layer was washed 2 times with water and then brine and dried over magnesium sulfate. Purification by silica flash chromatography using hexane/ethyl acetate mixture (9:1) as an eluent gave 2-(5′-fluoro-5-formyl-6-hydroxy-2′-methoxymethoxy-biphenyl-3-yl)-3-methoxy-2-methoxymethyl-propionic acid methyl ester (0.056 g, 61%).

Proceeding as in Reference 7, but substituting 2-(4-benzyloxy-3-bromo-5-formylphenyl)-3-hydroxy-2-hydroxymethyl-propionic acid methyl ester, prepared as in Reference 5, and 5-fluoro-2-methoxymethoxy-benzeneboronic acid gave 2-(6-benzyloxy-5′-fluoro-5-formyl-2′-methoxymethoxy-biphenyl-3-yl)-3-hydroxy-2-hydroxymethyl-propionic acid methyl ester (0.07 g, 58%).

Proceeding as in Reference 7, but substituting [3-bromo-5-formyl-4-(2-methoxyethoxymethoxy)-phenyl]-acetic acid methyl ester and 4-(2-methoxy-ethoxymethoxy)-3-(4,4,5,5-tetramethyl-[1,3,2]dioxaborolan-2-yl)-benzonitrile gave [5′-cyano-5-formyl-6,2′-bis-(2-methoxy-ethoxymethoxy)-biphenyl-3-yl]-acetic acid methyl ester.

Proceeding as in Reference 13, but substituting 2-(3-bromo-5-formyl-4-hydroxyphenyl)-3-hydroxy-2-hydroxymethyl-propionic acid methyl ester, gave 2-(5′-fluoro-5-formyl-6-hydroxy-2′-methoxymethoxy-biphenyl-3-yl)-3-hydroxy-2-hydroxymethyl-propionic acid methyl ester.

### Reference 8

### Synthesis of 2-(4-hydroxyphenyl)succinic acid dimethyl ester

2-(4-Methoxyphenyl)-succinic acid dimethyl ester (5.0 g, 20 mmol) was heated with 48% aqueous hydrogen bromide (50 mL) for 16 hours. The reaction mixture was cooled to form a precipitate. The precipitate was filtered, washed with water and dried in high vacuum over phosphorus pentoxide overnight to give 2-(4-hydroxyphenyl)succinic acid.

The 2-(4-hydroxyphenyl)succinic acid was dissolved in methanol (150 mL) and thionyl chloride (2 mL) was added dropwise to the solution. The reaction mixture was stirred overnight, the solvent was evaporated and the residue was partitioned between ethyl ether and 5% aqueous sodium bicarbonate. The ether layer was washed with water, brine and dried over magnesium sulfate. The solvent was evaporated to give 2-(4-hydroxyphenyl)succinic acid dimethyl ester (2.4 g).

### Reference 9

### Synthesis of 2-[4-(2-methoxy-ethoxymethoxy)-benzylidene]-malonic acid dimethyl ester

A mixture of 2-(4-hydroxybenzylidene)malonic acid dimethyl ester (5.9 g, 25 mmol), dichloromethane and diisopropylethylamine (3.9 g, 30 mmol) was cooled in an ice bath. MEM-Cl (3.1 g, 25 mmol) was added and the resulting mixture was stirred overnight at ambient temperature. The reaction mixture was concentrated and the residue was partitioned between citric acid and ether. The ether layer was washed with 1N aqueous sodium hydroxide, water, citric acid, water, and brine. The organic layer was separated and concentrated to give 2-[4-(2-methoxy-ethoxymethoxy)-benzylidene]-malonic acid dimethyl ester.

Proceeding as in Reference 9, but substituting 2-(4-hydroxyphenyl)succinic acid dimethyl ester, prepared as in Reference 8, gave 2-[4-(2-methoxy-ethoxymethoxy)-phenyl]-succinic acid dimethyl ester.

### Reference 10

### Synthesis of 3-(4-hydroxyphenyl)-tetrahydrofuran-2-one

2-[4-(2-Methoxy-ethoxymethoxy)-benzylidene]-malonic acid (6.5 g, 20 mmol), prepared as in Reference 9, was combined with potassium cyanide (1.43 g, 22 mmol) in ethanol containing 5% of water and the mixture was refluxed for 24 hours. The reaction mixture was poured into 5% aqueous citric acid and extracted with diethyl ether. The ether layer was washed with water and then brine and concentrated to give 3-cyano-3-[4-(2-methoxy-ethoxymethoxy)-phenyl]-propionic acid.

Methyl iodide (1.7 g, 12 mmol) was added to a mixture of the 3-cyano-3-[4-(2-methoxy-ethoxymethoxy)-phenyl]-propionic acid (2.9 g, 10.4 mmol) and cesium carbonate (3.6 g, 11 mmol), in dimethylformamide (20 mL) and the mixture was stirred overnight. The reaction mixture was poured into water and extracted with diethyl ether. The extracts were washed with water and then brine and concentrated to give 3-cyano-3-[4-(2-methoxyethoxymethoxy)-phenyl]-propionic acid methyl ester.

The 3-cyano-3-[4-(2-methoxy-ethoxymethoxy)-phenyl]-propionic acid methyl ester (2.65 g, 9.0 mmol) was dissolved in THF and the solution heated to −78° C. A solution of diisobutylaluminum (20 mL, 1M in hexanes, 20 mmol) was cooled to 0° C. and n-butyl lithium (8 mL, 2.5 M in hexane, 20 mmol) was added. The n-butyl lithium mixture was stirred for 20 minutes and then slowly transferred to the solution of propionic acid methyl ester. The mixture allowed stand for 3 hours and then sodium borohydride (1 g) in ethanol (25 mL) was added. The mixture was stirred until the reaction was complete, then poured in cold 3% hydrochloric acid, extracted with ethyl acetate and concentrated to give 3-cyano-3-[4-(2-methoxy-ethoxymethoxy)-phenyl]-propan-1-ol.

The 3-cyano-3-[4-(2-methoxy-ethoxymethoxy)-phenyl]-propan-1-ol (2.1 g, 8 mmol) was combined with concentrated sulfuric acid (1.6 g, 16 mmol) in water (20 mL) and the mixture was refluxed for 24 hours. The reaction mixture was worked up and the crude product was purified to give 3-(4-hydroxyphenyl)-tetrahydrofuran-2-one.

### Reference 11

### Synthesis of acetic acid 4-acetoxy-3-(4-hydroxy-phenyl)-butyl ester

2-[4-(2-methoxy-ethoxymethoxy)-phenyl]-succinic acid dimethyl ester (2.31 g, 7.1 mmol), prepared as in Reference 9, in was dissolved in THF and lithium aluminum hydride (30 ml, 1 N in THF, 30 mmol) was added. The reaction mixture was stirred at room temperature for 2 hours and then poured into water. The mixture was extracted with ethyl ether and the organic layer was washed with water and then brine and dried over magnesium sulfate. The solvent was evaporated to give 4-hydroxy-2-[4-(2-methoxyethoxy-methoxy)-phenyl]-butan-1-ol (2.0 g).

The 4-hydroxy-2-[4-(2-methoxyethoxy-methoxy)-phenyl]-butan-1-ol (2.0 g, 7.0 mmol) and pyridine (1.6 g, 20 mmol) were dissolved in benzene (40 mL) and then acetyl chloride (1.05 mL, 16.8 mmol) was added to the mixture over 10 min. The mixture was stirred for 1 hour and then partitioned between 5% aqueous citric acid and ethyl ether. The organic layer was separated and washed with water and then brine and dried over magnesium sulfate. The organic layer was concentrated to give acetic acid 4-acetoxy-3-[4-(2-methoxy-ethoxymethoxy)-phenyl]-butyl ester (2.1 g).

The acetic acid 4-acetoxy-3-[4-(2-methoxy-ethoxymethoxy)-phenyl]-butyl ester (2.1 g, 6.0 mmol) was dissolved in dichloromethane (25 mL) and trifluoroacetic acid (10 mL) was added. The reaction mixture was stirred for 4 hours and the solvents were evaporated in vacuum. The residue was partitioned between ethyl ether and water and the organic layer was separated washed with water and then brine and dried over magnesium sulfate. The organics were removed in vacuum to give acetic acid 4-acetoxy-3-(4-hydroxyphenyl)-butyl ester (1.1 g).

### Reference 12

### Synthesis of 3-(3-bromo-5-formyl-4-hydroxyphenyl)-tetrahydrofuran-2-one

3-(4-Hydroxyphenyl)tetrahydrofuran-2-one (0.37 g, 2.1 mmol), prepared as in Reference 10, was combined with dry acetonitrile and the mixture was treated with anhydrous magnesium chloride (0.315 g, 4.75 mmol), triethylamine (TEA) (0.84 g, 0.14 mmol) and paraformaldehyde (0.66 g, 23 mmol) under refluxed for approximately 1 hour. The mixture was cooled to ambient temperature and mixed with 1N HCl in ether. The organic layer was isolated and the aqueous layer was extracted with ether. The combined organic extracts were dried (MgSO₄) and concentrated under reduced pressure to yield 3-(3-formyl-4-hydroxyphenyl)tetrahydrofuran-2-one.

The 3-(3-formyl-4-hydroxyphenyl)tetrahydrofuran-2-one (0.185 g, 0.9 mmol) was combined with dry DMF and the mixture was diluted, in a drop wise manner, with a solution of N-bromosuccinimide (NBS, 1.19 g, 1.09 mmol) in DMF. The mixture was agitated for about 2 hours and then concentrated under reduced pressure at less than 35° C. The residue was dissolved in ether and the mixture was washed with water. The ether layer was dried (MgSO₄) and then concentrated and purified by column chromatography to give 3-(3-bromo-5-formyl-4-hydroxyphenyl)-tetrahydrofuran-2-one (0.14 g).

Proceeding as in Reference 12, but substituting acetic acid 4-acetoxy-3-(4-hydroxyphenyl)-butyl ester, prepared as in Reference 11, gave acetic acid 4-acetoxy-3-(3-bromo-5-formyl-4-hydroxy-phenyl)-butyl ester.

### Reference 13

### Synthesis of 3-(5′-fluoro-5-formyl-2′,6-dihydroxy-biphenyl-3-yl)-tetrahydrofuran-2-one

3-(3-Bromo-5-formyl-4-hydroxyphenyl)-tetrahydrofuran-2-one (0.12 g, 0.42 mmol), prepared as in Reference 12, was combine with 5-fluoro-2-methoxyethoxyphenyl-boronic acid (0.105 g, 0.53 mmol), 2M Na₂CO₃ (0.3 mL, 0.6 mmol) and tetrakis-(triphenylphosphine) palladium (24 mg, 0.021 mmol) in ethylene glycol dimethyl ether (10 mL). The mixture was put under an atmosphere of nitrogen, stirred and heated to reflux for 4 hours. The mixture was neutralized with 1N hydrochloric acid and extracted with ethyl acetate. The organic layer was dried (MgSO₄), filtered and concentrated via rotoevaporation to a gum. Product was purified from the residue by chromatography on silica gel to give 3-(5′-fluoro-5-formyl-6-hydroxy-2′-methoxyethoxy-biphenyl-3-yl)-tetrahydrofuran-2-one.

The 3-(5′-fluor Q-5-formyl-6-hydroxy-2′-methoxyethoxy-biphenyl-3-yl)-tetrahydrofuran-2-one was dissolved in dichloromethane (5 mL) and trifluoroacetic acid (1 mL) was added. The mixture was stirred for 2 hours and concentrated to give 3-(5′-fluoro-5-formyl-2′,6-dihydroxy-biphenyl-3-yl)-tetrahydrofuran-2-one.

Proceeding as in Reference 13, but substituting acetic acid 4-acetoxy-3-(3-bromo-5-formyl-4-hydroxy-phenyl)-butyl ester, prepared as in Reference 12, gave acetic acid 4-acetoxy-3-(5′-fluoro-5-formyl-6,2′-dihydroxy-biphenyl-3-yl)-butyl ester.

### Example 1

### Synthesis of 3-[5-(5-carbamimidoyl-1H-benzoimidazol-2-yl)-5′-fluoro-2′6-dihydroxybiphenyl-3-yl]-tetrahydrofuran-2-one

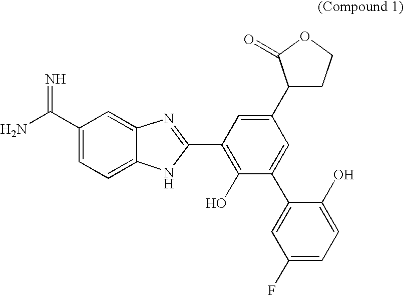

3-(5′-Fluoro-5-formyl-2′,6-dihydroxy-biphenyl-3-yl)-tetrahydrofuran-2-one (0.1 g, 0.32 mmol), prepared as in Reference 13, was combined with 3,4-diaminobenzamidine hydrochloride (90 mg, 0.48 mmol) and benzoquinone (34 mg, 0.32 mmol) in ethanol and the mixture heated for approximately 30 minutes. The reaction mixture was cooled to ambient temperature and concentrated under reduced pressure. The residue was purified by reverse phase HPLC to yield 3-[5-(5-carbamimidoyl-1H-benzoimidazol-2-yl)-5′-fluoro-2,6-dihydroxy-biphenyl-3-yl]tetrahydrofuran-2-one. MS: found (M+H) 447.3, (M−H+) 445.4; calcd. 446.14.

Proceeding as in Example 1, but substituting acetic acid 4-acetoxy-3-(5′-fluoro-5-formyl-6,2′-dihydroxy-biphenyl-3-yl)-butyl ester, prepared as in Reference 13, gave acetic acid 4-acetoxy-3-[5-(5-carbamimidoyl-1H-benzoimidazol-2-yl)-5′-fluoro-6,2′-dihydroxybiphenyl-3-yl]-butyl ester hydrochloride (Compound 2).

### Example 2

### Synthesis of 2-[5-(5-carbamimidoyl-1H-benzoimidazol-2-yl)-5′-fluoro-2′6-dihydroxybiphenyl-3-yl]4-hydroxybutanoic acid

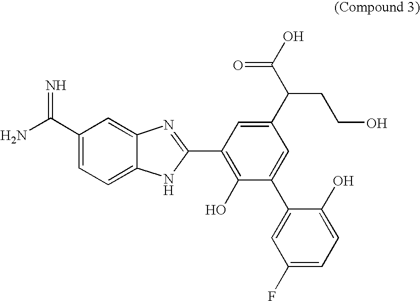

3-[5-(5-Carbamimidoyl-1H-benzoimidazol-2-yl)-5′-fluoro-2,6-dihydroxy-biphenyl-3-yl]tetrahydrofuran-2-one (28 mg), prepared as in Example 1, was dissolved in methanol (2 mL) and 1 N aqueous sodium hydroxide was added. The reaction mixture was neutralized and the product was extracted and the crude was purified by HPLC to give 2-[5-(5-carbamimidoyl-1H-benzoimidazol-2-yl)-5′-fluoro-2′6-dihydroxy-biphenyl-3-yl]-4-hydroxybutanoic acid. MS: found (M+H⁺) 465.3, calc. 464.15.

### Example 3

### Synthesis of 2-[5-(5-carbamimidoyl-1H-benzoimidazol-2-yl)-5′-fluoro-2′6-dihydroxybiphenyl-3-yl]-4-hydroxybutan-1-ol

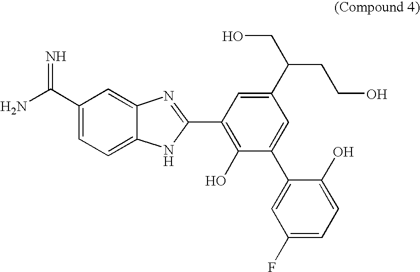

Acetic acid 4-acetoxy-3-[5-(5-carbamimidoyl-1H-benzoimidazol-2-yl)-5′-fluoro-6,2′-dihydroxy-biphenyl-3-yl]-butyl ester hydrochloride (0.105 g, 0.18 mmol), prepared as in Example 1, was combined with acetonitrile (110 mL) and 3N aqueous hydrogen chloride (10 mL) and the mixture was refluxed for 1 hour. The solvents were evaporated in vacuum and the crude product was subjected to HPLC purification (C-18, acetonitrile gradient) to give 2-[5-(5-carbamimidoyl-1H-benzoimidazol-2-yl)-5′-fluoro-2′6-dihydroxy-biphenyl-3-yl]4-hydroxybutan-1-ol (80 mg). ¹H NMR (CDCl₃): δ 1.62-1.69 (m, 1H), 1.87-1.94 (m, 1H), 2.73-2.79 (m, 1H), 3.22-3.35 (m, 2H), 3.51-3.56 (m, 2H), 6.83-7.87 (m, 1H), 6.92-6.98 (m, 1H), 7.16 (d, J=1.8 Hz, 1H), 7.67(d, J=8.3 Hz, 1H), 7.77 (d, J=8.3 Hz, 1H), 7.98 (s, 1H), 8.11 (s, 1H), 9.02 (s, 2H), 9.32 (s, 2H). MS: found (M+H⁺) 451.2, (M−H⁺) 449.3, calc. 450.17.

### Example 4

### Synthesis of 2-[5-(5-Carbamimidoyl-1H-benzoimidazol-2-yl)-5′-fluoro-6,2′-dihydroxybiphenyl-3-yl]-3-hydroxy-2-hydroxymethyl-propionic acid

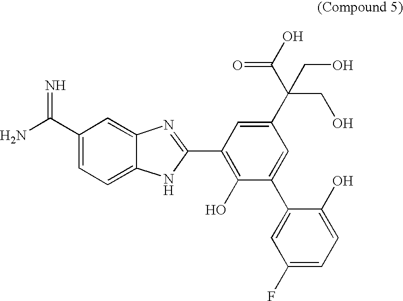

2-(6-Benzyloxy-5′-fluoro-5-formyl-2′-methoxymethoxy-biphenyl-3-yl)-3-hydroxy-2-hydroxymethyl-propionic acid methyl ester (0.087 g, 0.18 mmol), prepared as in Reference 7, was combined with 3,4-diaminobenzamidine hydrochloride (0.04 g, 0.22 mmol) and 1,4-benzoquinone (0.02 g, 0.18 mmol) in methanol (15 ml) and the mixture was heated under reflux for 3 hours. The solvent was evaporated in vacuum and the residue was triturated with ethyl ether and recovered by filtration to give 2-[6-benzyloxy-5-(5-carbamimidoyl-1H-benzoimidazol-2-yl)-5′-fluoro-2′-methoxymethoxy-biphenyl-3-yl]-3-hydroxy-2-hydroxymethyl-propionic acid methyl ester (0.11 g).

The 2-[6-benzyloxy-5-(5-carbamimidoyl-1H-benzoimidazol-2-yl)-5′-fluoro-2′-methoxymethoxy-biphenyl-3-yl]-3-hydroxy-2-hydroxymethyl-propionic acid methyl ester was dissolved in methanol (6 ml) and the solution treated with hydrogen chloride (3 ml, 4N in dioxane) for 6 hours. The solvents were removed in vacuum and the residue triturated with ethyl ether and recovered by filtration to give 2-[6-benzyloxy-5-(5-carbamimidoyl-1H-benzoimidazol-2-yl)-5′-fluoro-2′-hydroxy-biphenyl-3-yl]-3-hydroxy-2-hydroxymethyl-propionic acid methyl ester (0.1 g).

The 2-[6-benzyloxy-5-(5-carbamimidoyl-1H-benzoimidazol-2-yl)-5′-fluoro-2′-hydroxy-biphenyl-3-yl]-3-hydroxy-2-hydroxymethyl-propionic acid methyl ester was heated with aqueous hydrochloric acid (3 ml, 3N) in acetonitrile (3 ml) for 12 hours. The solvents were removed in vacuum and product was purified from the residue by reversed phase HPLC (acetonitrile gradient) to give 2-[5-(5-carbamimidoyl-1H-benzoimidazol-2-yl)-5′-fluoro-6,2′-dihydroxy-biphenyl-3-yl]-3-hydroxy-2-hydroxymethyl-propionic acid (0.03 g). ¹H NMR (DMSO-d₆): δ 3.94(d, J=10 Hz, 2H), 4.03 (d, J=10 Hz, 2H), 6.83-6.97 (m, 3H), 7.19(s, 1H), 7.65 (d, J=8.4 Hz, 1H), 7.77 (d, J=8.4 Hz, 1H), 7.89 (d, J=2.1 Hz, 1H), 8.11 (s, 1H), 8.90 (s, 2H), 9.24 (s, 2H). MS: found (M+H⁺) 481.1, (M−H⁺) 478.9, calcd. 480.14.

Proceeding as in Example 4, but substituting 2-(5′-cyano-5-formyl-6,2′-bis(2-methoxyethoxymethoxy)-biphenyl-3-yl)-3-hydroxy-2-hydroxymethyl-propionic acid methyl ester, gave 2-[5-(5-carbamimidoyl-1H-benzoimidazol-2-yl)-5′-cyano-6,2′-dihydroxybiphenyl-3-yl]-3-hydroxy-2-hydroxymethyl-propionic acid methyl ester (Compound 6).

Proceeding as in Example 4, but substituting 2-(5′-fluoro-5-formyl-6-hydroxy-2′-methoxymethoxy-biphenyl-3-yl)-3-methoxy-2-methoxymethyl-propionic acid methyl ester, gave 2-[5-(5-carbamimidoyl-1H-benzoimidazol-2-yl)-5′-fluoro-6,2′-dihydroxy-biphenyl-3-yl]-3-methoxy-2-methoxymethyl-propionic acid (Compound 7). ¹H NMR (DMSO-d₆): 3.36(s, 6H), 3.98 (d,J=8.5 Hz, 2H), 4.05 (d,J=8.5 Hz, 2H) 6.96-7.20 (m, 3H), 7.32(d,J=2.1 Hz, 1H), 7.80 (d,J=8.4 Hz, 1H), 7.91 (d,J=8.4 Hz, 1H), 8.04 (d,J=2.1 Hz, 1H), 8.23(s, 1H), 9.11 (s, 2H), 9.43 (s, 2H). MS: found (M+H⁺) 509.2, (M−H⁺) 507.0, calc. 508.18.

### Example 5

### Synthesis of 2-[5-(5-carbamimidoyl-1H-benzoimidazol-2-yl)-6,2′-dihydroxy-5′-ureidomethyl-biphenyl-3-yl]-3-hydroxy-2-hydroxymethyl-propionic acid

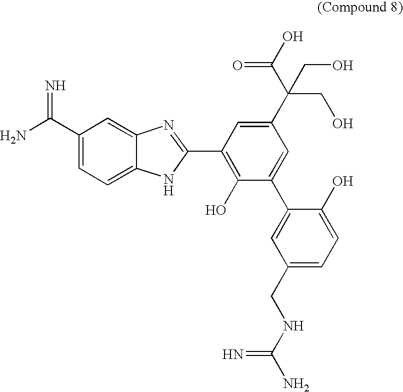

2-[5-(5-Carbamimidoyl-1H-benzoimidazol-2-yl)-5′-cyano-6,2′-dihydroxy-biphenyl-3-yl]-3-hydroxy-2-hydroxymethyl-propionic acid methyl ester (0.108 g, 0.2 mmol), prepared as in Example 4, was dissolved in aqueous hydrochloric acid (3 ml, 1N) and methanol (6 ml) and the solution was subjected to hydrogenation at 1 atm over Pd(OH)₂ (0.03 g) for 2 hours. The mixture was filtered through celite and the filtrate was concentrated in vacuum. The residue was triturated with ethyl ether and the solids were collected by filtration to give 2-[5′-aminomethyl-5-(5-carbamimidoyl-1H-benzoimidazol-2-yl)-6,2′-dihydroxy-biphenyl-3-yl]-3-hydroxy-2-hydroxymethyl-propionic acid methyl ester (0.1 g).

The 2-[5′-aminomethyl-5-(5-carbamimidoyl-1H-benzoimidazol-2-yl)-6,2′-dihydroxy-biphenyl-3-yl]-3-hydroxy-2-hydroxymethyl-propionic acid methyl ester was heated with aqueous hydrochloric acid (6 ml, 3N) in acetinitrile (6 ml) for 6 hours. The solvents were removed in vacuum and product purified from the residue by reversed phase HPLC (acetonitrile gradient) to give 2-[5′-aminomethyl-5-(5-carbamimidoyl-1H-benzoimidazol-2-yl)-6,2′-dihydroxy-biphenyl-3-yl]-3-hydroxy-2-hydroxymethyl-propionic acid (0.09 g).

The 2-[5′-aminomethyl-5-(5-carbamimidoyl-1H-benzoimidazol-2-yl)-6,2′-dihydroxy-biphenyl-3-yl]-3-hydroxy-2-hydroxymethyl-propionic acid was dissolved in a mixture of methanol and water and then triethylamine was added to establish pH 9. Potassium cyanate (0.081 g, 1 mmol) was added and the mixture was heated for 12 hours at 45 to 55° C. The mixture was concentrated under reduced pressure and the product was purified from the residue by reversed phase HPLC (acetonitrile gradient) to give 2-[5-(5-carbamimidoyl-1H-benzo imidazol-2-yl)-6,2′-dihydroxy-5′-ureidomethyl-biphenyl-3-yl]-3-hydroxy-2-hydroxymethyl-propionic acid. MS: found (M+H⁺) 535.1, (M−H+) 533.3, calc. 534.19.

Proceeding as in Example 5, but substituting sulfamide for potassium cyanate, gave 2-[5-(5-carbamimidoyl-1H-benzoimidazol-2-yl)-6,2′-dihydroxy-5′-aminosulfonylaminomethyl-biphenyl-3-yl]-3-hydroxy-2-hydroxymethyl-propionic acid (Compound 9).

### Biological Examples

### Example 6

### In Vitro Factor VIIa Inhibitor Assay

Mixtures of human Factor VIIa (0.5-5 nM) and test compound (varying concentrations) in assay medium (comprising: NaCl, 150 mM (pH 7.4); CaCl₂, 5 mM; Tween-20, 0.05%; tissue factor, 25 nM; EDTA, 1.5 mM; and dimethylsulfoxide, 10%). Human Factor VIIa (7 nM) incubated with the substrate (500 μM of CH₃SO₂-D-CHA-But-Arg-pNA) in the presence of inhibitor. Hydrolysis of the chromogenic substrate was followed spectrophotometrically at 405 nm for five minutes. Initial velocity measurements calculated from the progress curves by a kinetic analysis program (Batch Ki; Peter Kuzmic, BioKin, Ltd., Madison, Wis.) were used to determine the Kᵢ app.

Compounds of the invention tested by the above-described assay exhibited inhibition of Factor VIIa.

### Example 7

### In Vitro Factor Xa Inhibitor Assay

Mixtures of human Factor Xa (0.5-5 nM) and test compound (varying concentrations) in assay medium (comprising: Tris, 50 mM (pH 8); NaCl, 1M; Ca:Cl₂, 5 mM; polyoxyethylenesorbitan monolaurate (Tween-20), 0.05%; DMSO, 10%; zinc chloride, 150 μM) were incubated for 1 hour at room temperature and then substrate, MesOC-Norleu-Gly-Arg-pNA, was added such that the final concentration of the substrate in the assay mixture was between 0.5 and 5 mM. Hydrolysis of the substrate was followed spectrophotometrically at (405λ) for 5 minutes. Apparent inhibition constants (Kᵢ) were calculated from the enzyme progress curves using standard mathematical models.

Compounds of the invention tested by the above-described assay exhibited inhibition of Factor Xa.

### Example 8

### Pharmacokinetic Assay

Rats with pre-implanted jugular vein catheters, filled with heparin/saline/PVP lock prior to shipment, were obtained from Charles River. Three rats were selected for each study, weighed and administered test compound by tail vein injection. Any residual test compound was retained and stored at −70° C. for later analysis.

Blood samples (0.25 mL each) were collected from the indwelling catheters at specified times over 120 hours. The catheters were flushed with physiological saline immediately after each collection and filled with heparinized saline after each 8, 24 and 48 hour collection. In the event that a catheter failed, blood samples were collected via the retro-orbital sinus under isoflurane anesthesia at the appropriate time.

Blood samples were placed in 0.5 mL Microtainer® tubes (lithium heparin), shaken gently and stored on wet ice. The samples were centrifuged for 10 minutes at 2400 rpm in a refrigerated centrifuged. Plasma samples (100 μL) from each tube were transferred to 0.5 mL Unison polypropylene vials (Sun-500210) and stored below −70° C. for later analysis by LC/MS-MS.

### Example 9

### In Vitro Clotting Assays

Coagulation assays, activated partial thromboplastin time (aPTT) and prothrombin time (PT) were carried out based on the procedure described in Hougie, C. Hematology (Williams, W. J., Beutler, B., Erslev, A. J., and Lichtman, M. A., Eds.), pp. 1766-1770 (1990), McGraw-Hill, New York.

Briefly, the assays were performed using normal human citrated plasma and were performed at 37° C. on a coagulometer (Electra 800) in accordance with the manufacturer's instructions (Medical Laboratory Automation—Pleasantville, N.Y.). The instrument was calibrated with plasma immediately prior to collecting clotting times for samples with inhibitors. The aPTT and PT doubling concentrations were calculated by fitting inhibitor dose response curves to a modified version of the Hill equation.

### Example 10

### Pharmaceutical Compositions

The following are representative pharmaceutical formulations containing a compound of Formula I.

### Tablet Formulation

The following ingredients are mixed intimately and pressed into single scored tablets.

| | Quantity per |
|---|---|
| Ingredient | tablet, mg |
| compound of this invention | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| | Quantity per |
|---|---|
| Ingredient | capsule, mg |
| compound of this invention | 200 |
| lactose, spray-dried | 148 |
| magnesium stearate | 2 |

### Suspension Formulation

The following ingredients are mixed to form a suspension for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.5 | g |
| sorbitol (70% solution) | 12.85 | g |
| Veegum K (Vanderbilt Co.) | 1.0 | g |
| flavoring | 0.035 | ml |
| colorings | 0.5 | mg |
| distilled water | q.s. to 100 | ml |

### Injectable Formulation

The following ingredients are mixed to form an injectable formulation.

| Ingredient | Amount |
|---|---|
| compound of this invention | 1-2 g |
| sodium acetate buffer solution, | 0.4 M 2.0 ml |
| HCl (1 N) or NaOH (1 N) | q.s. to suitable pH |
| water (distilled, sterile) | q.s. to 20 ml |

All of the above ingredients, except water, are combined and heated to 60-70.degree. C. with stirring. A sufficient quantity of water at 60.degree. C. is then added with vigorous stirring to emulsify the ingredients, and water then added q.s. to 100 g.

### Suppository Formulation

A suppository of total weight 2.5 g is prepared by mixing the compound of the invention with Witepsol.RTM. H-15 (triglycerides of saturated vegetable fatty acid; Riches-Nelson, Inc., New York), and has the following composition:

| | |
|---|---|
| compound of the invention | 500 mg |
| Witepsol .RTM. H-15 | balance |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A compound of Formula I: 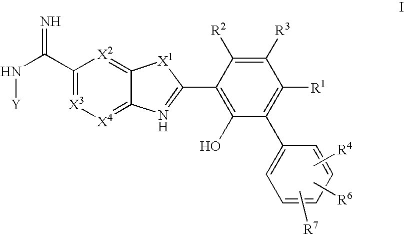
wherein:
X¹, X², X³ and X⁴ are independently —N— or —CR⁵—, wherein R⁵ is hydrogen, alkyl or halo, with the proviso that not more than three of X¹, X², X³ and X⁴ are —N—;
R¹ and R² independently are hydrogen, alkyl, hydroxyalkyl or halo;
R³ is:
(xiii) hydroxyalkyl;
(xiv) hydroxyalkyl substituted with alkoxy, carboxy, formyl, tetrazol-5-yl or alkoxycarbonyl;
(xv) carboxyalkyl substituted with tetrazol-5-yl, aryloxycarbonyl, alkylaminoalkoxycarbonyl, dialkylaminoalkoxycarbonyl, one to two alkoxy groups, one to four halo, amino, alkylamino, dialkylamino, oxo, —(OCH₂CH₂O)ₙ₁—OR (wherein n1 is an integer from 1 to 3 and R is hydrogen or alkyl) or —NR^{a}R^{b}, wherein R^{a} is hydrogen or alkyl and R^{b} is acyl or —SO₂R^{c}, wherein R^{c} is alkyl);
(xvi) cycloalkyl substituted with carboxy or carboxyalkyl;
(xvii) carboxyalkyloxy or dicarboxyalkyloxy; or
(xviii) a group of formula (a), (b), (c), (d) or (e): 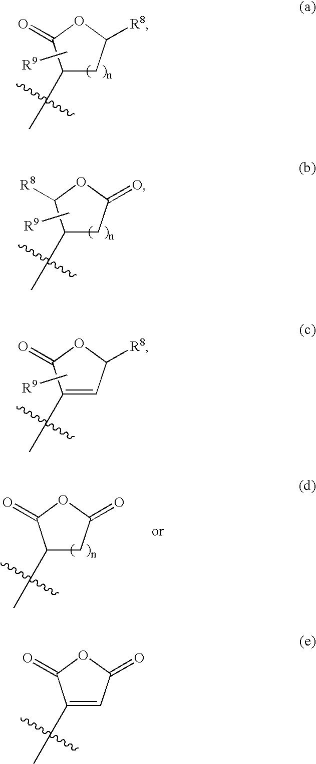
wherein:
n is 1 or 2;
R⁸ is hydrogen, alkyl, alkoxy or hydroxy; and
R⁹ is hydrogen or alkyl;
R⁴ is hydrogen, alkyl, alkylthio, halo, hydroxy, hydroxyalkyl, alkoxy, aminosulfonyl, alkylaminosulfonyl, dialkylaminosulfonyl or nitro;
R⁶ is hydrogen, alkyl or halo;
R⁷ is hydrogen, alkyl, haloalkyl, cycloalkyl, alkylthio, halo, hydroxy, hydroxyalkyl, nitro, cyano, alkoxy, alkoxyalkyl, alkoxyalkyloxy, hydroxyalkyloxy, aminoalkyloxy, carboxyalkyloxy, aminocarbonylalkyloxy, haloalkoxy, carboxy, carboxyalkyl, alkoxycarbonyl, alkoxycarbonylalkyl, cyanoalkyl, alkylsulfonyl, alkylsulfonylalkyl, arylsulfonyl, heteroarylsulfonyl, carbamimidoyl, hydroxycarbamimidoyl, alkoxycarbamimidoyl, alkylsulfonylamino, alkylsulfonylaminoalkyl, alkoxysulfonylamino, alkoxysulfonylaminoalkyl, heterocycloalkylalkylaminocarbonyl, hydroxyalkoxyalkylaminocarbonyl, heterocycloalkylcarbonyl, heterocycloalkylcarbonylalkyl, heterocycloalkyl, heterocycloalkylalkyl, oxoheterocycloalkyl, oxoheterocycloalkylalkyl, heteroaryl, heteroaralkyl, ureido, alkylureido, dialkylureido, ureidoalkyl, alkylureidoalkyl, dialkylureidoalkyl, thioureido, thioureidoalkyl, acyldifluoromethyl, —COR¹² (wherein R¹² is alkyl, haloalkyl, hydroxyalkyl, alkoxyalkyl, oxoalkyl, dialkylaminocarbonylalkyl, alkoxycarbonylalkyl, cyanoalkyl or aminoalkyl), -(alkylene)-COR¹² (wherein R¹² is alkyl, haloalkyl, hydroxyalkyl, alkoxyalkyl or aminoalkyl), —CONR¹⁴R¹⁵ (wherein R¹⁴ is hydrogen or alkyl and R¹⁵ is hydrogen, alkyl, hydroxyalkyl, alkoxyalkyl, aryl, aralkyl, heteroaryl or heteroaralkyl), -(alkylene)-CONR¹⁶R¹⁷ (wherein R¹⁶ is hydrogen, alkyl or hydroxyalkyl and R¹⁷ is hydrogen, alkyl, hydroxyalkyl, alkoxyalkyl, aryl, aralkyl, heteroaryl or heteroaralkyl), —NR¹⁸R¹⁹ (wherein R¹⁸ is hydrogen or alkyl and R¹⁹ is hydrogen, alkyl, acyl, aryl, aralkyl, heteroaryl or heteroaralkyl), -(alkylene)-NR²⁰R²¹ (wherein R²⁰ is hydrogen, alkyl or hydroxyalkyl and R²¹ is hydrogen, alkyl, acyl, alkoxycarbonyl, hydroxyalkyl, alkoxyalkyl, aryl, aralkyl, heteroaryl or heteroaralkyl), —SO₂NR²²R²³ (wherein R²² is hydrogen or alkyl and R²³ is hydrogen, alkyl, aryl, aralkyl, heteroaryl or heteroaralkyl or R²² and R²³ together with the nitrogen atom to which they are attached from heterocycloamino), -(alkylene)-SO₂NR²⁴R²⁵ (wherein R²⁴ is hydrogen or alkyl and R²⁵ is hydrogen, alkyl, aryl, aralkyl, heteroaryl or heteroaralkyl or R²⁴ and R²⁵ together with the nitrogen atom to which they are attached from heterocycloamino), —NR²⁶SO₂NR²⁷R²⁸ (wherein R²⁶ and R²⁷ are independently hydrogen or alkyl and R²⁸ is hydrogen, alkyl, aryl, aralkyl, heteroaryl or heteroaralkyl or R²⁷ and R²⁸ together with the nitrogen atom to which they are attached from heterocycloamino), -(alkylene)-NR²⁹SO₂NR³⁰R³¹ (wherein R²⁹ and R³⁰ are independently hydrogen or alkyl and R³¹ is hydrogen, alkyl, aryl, aralkyl, heteroaryl or heteroaralkyl or R³⁰ and R³¹ together with the nitrogen atom to which they are attached from heterocycloamino), —CONH-(alkylene)-NR³²R³³ (wherein R³² is hydrogen or alkyl and R³³ is alkyl) or aralkyl; and
Y is hydrogen, hydroxy, alkoxy, haloalkoxy, haloalkoxycarbonyl, —C(O)R³⁵ (wherein R³⁵ is alkyl, aryl, haloalkyl, or cyanoalkyl) or —C(O)OR³⁶ (wherein R³⁶ is alkyl, hydroxyalkyl, alkoxyalkyl, alkoxycarbonylalkyl, acyl, aryl or haloalkyl); and individual stereoisomers or mixture of stereoisomers; or a pharmaceutically acceptable salt thereof; provided that when R³ is: (i) carboxyalkyl substituted with aryloxycarbonyl, one to two alkoxy groups, one to four halo, amino, alkylamino, dialkylamino or oxo or (ii) cycloalkyl substituted with carboxy and R⁷ is hydrogen, alkyl, haloalkyl, halo, nitro, alkoxy, haloalkyl, carboxy, alkoxycarbonyl, —NR¹⁸R¹⁹ (wherein R¹⁸ is hydrogen or alkyl and R¹⁹ is hydrogen, alkyl, aryl or aralkyl), pyrrolidinylcarbonyl, —SO₂NR²²R²³ (wherein R²² and R²³ are alkyl), carbamimidoyl, alkylsulfonylamino, alkylthio, ureido, —NHC(S)NH₂ or heterocycloamino, then R⁴ is hydroxy or hydroxyalkyl.

2. The compound of claim 1 wherein X¹ is —N— and X², X³ and X⁴ are —CR⁵—, wherein R⁵ is hydrogen.

3. The compound of claim 1 wherein X¹ is —N—; X² and X⁴ are —CR⁵—, wherein R⁵ is hydrogen, and X³ is —CR⁵—, wherein R⁵ is halo.

4. The compound of claim 1 wherein X¹ is —C=and X², X³ and X⁴ are —CR⁵—, wherein R⁵ is hydrogen.

5. The compound of claim 1 wherein X¹ is —C═, X² and X⁴ are —CR⁵—, wherein R⁵ is hydrogen, and X³ is —CR⁵—, wherein R⁵ is halo.

6. The compound of claim 2 wherein Y, R¹ and R² each are hydrogen and R⁴ is hydroxy and is located at the 2′-position of the biphenylyl moiety.

7. The compound of claim 6 wherein R⁶ is hydrogen and R⁷ is located at the 5′-position of the biphenylyl moiety and is alkyl, halo, hydroxy, hydroxyalkyl, carboxy, alkoxy, cyano, nitro, aminocarbonyl, alkylsulfonylamino, aminoalkyl, ureidoalkyl, ureido, aminosulfonylaminoalkyl, cyanoalkyl, carboxyalkyl, aminocarbonylalkyl, acyldifluoromethyl, oxoalkyl, dialkylaminocarbonylalkyl, alkoxycarbonylalkyl, cyanoalkyl or heteroaryl.

8. The compound of claim 7 wherein R⁷ is methyl, isopropyl, chloro, fluoro, hydroxymethyl, carboxy, methoxy, cyano, nitro, aminocarbonyl, methylsulfonylamino, aminomethyl, ureidomethyl, imidazol-2-yl, amino, ureido, 2-cyanoethyl, carboxymethyl, 2-carboxyethyl, aminocarbonylmethyl or dimethylaminosulfonylamino.

9. The compound of claim 8 wherein R⁷ is fluoro or ureidomethyl.

10. The compound of claim 2 wherein R³ is 2-hydroxy-1-hydroxymethylethyl, 3-hydroxy-1-hydroxymethylpropyl, 1-carboxy-3-hydroxypropyl, 1-carboxy-2-methoxy-1-methoxymethylethyl, 1-carboxy-2-hydroxy-1-hydroxymethylethyl, 1-carboxy-1-hydroxymethyl-2-methoxyethyl, 2-oxo-tetrahydro-furan-3-yl, 5,5-dimethyl-2-oxo-tetrahydro-furan-3-yl, 2-oxo-2,5-dihydro-furan-3-yl, 2,5-dioxo-tetrahydro-furan-3-yl or 2,5-dioxo-2,5-dihydro-furan-3-yl.

11. The compound of claim 10 wherein R³ is 2-hydroxy-1-hydroxymethylethyl or 3-hydroxy-1-hydroxymethylpropyl.

12. The compound of claim 1 wherein:
Y, R¹ and R² each are hydrogen;
X¹ is —N— and X², X³, and X⁴ are —CR⁵—, wherein R⁵ is hydrogen;
R³ is 2-hydroxy-1-hydroxymethylethyl, 3-hydroxy-1-hydroxymethylpropyl, 1-carboxy-3-hydroxypropyl, 1-carboxy-2-methoxy-1-methoxymethylethyl, 1-carboxy-2-hydroxy-1-hydroxymethylethyl, 1-carboxy-1-hydroxymethyl-2-methoxyethyl, 2-oxo-tetrahydro-furan-3-yl, 5,5-dimethyl-2-oxo-tetrahydro-furan-3-yl, 2-oxo-2,5-dihydro-furan-3-yl, 2,5-dioxo-tetrahydro-furan-3-yl or 2,5-dioxo-2,5-dihydro-furan-3-yl;
R⁴ is hydroxy or hydroxymethyl and is located at the 2′-position of the biphenylyl moiety;
R⁶ is hydrogen; and
R⁷ is located at the 5′-position of the biphenylyl moiety and is alkyl, halo, hydroxy, hydroxyalkyl, carboxy, alkoxy, cyano, nitro, aminocarbonyl, alkylsulfonylamino, aminoalkyl, ureidoalkyl, ureido, aminosulfonylaminoalkyl, cyanoalkyl, carboxyalkyl, aminocarbonylalkyl, acyldifluoromethyl, oxoalkyl, dialkylaminocarbonylalkyl, alkoxycarbonylalkyl, cyanoalkyl or heteroaryl.

13. The compound of claim 12 wherein R⁷ is methyl, isopropyl, chloro, fluoro, hydroxymethyl, carboxy, methoxy, cyano, nitro, aminocarbonyl, methylsulfonylamino, aminomethyl, ureidomethyl, imidazol-2-yl, amino, ureido, 2-cyanoethyl, carboxymethyl, 2-carboxyethyl, aminocarbonylmethyl, dimethylaminosulfonylamino.

14. The compound of claim 13 wherein:
R³ is 3-hydroxy-1-hydroxymethylpropyl, 1-carboxy-3-hydroxypropyl, 1-carboxy-2-methoxy-1-methoxymethylethyl, 1-carboxy-2-hydroxy-1-hydroxymethylethyl, 1-carboxy-1-hydroxymethyl-2-methoxyethyl or 2-oxo-tetrahydro-furan-3-yl;
R⁴ is hydroxy; and
R⁷ is hydrogen, halo, ureidomethyl or aminosulfonylaminomethyl.

15. The compound of claim 12 wherein:
R³ is 1-carboxy-2-hydroxy-1-hydroxymethylethyl or 1-carboxy-3-hydroxypropyl; and
R⁷ is fluoro or ureidomethyl.

15. A pharmaceutical composition comprising a pharmaceutically acceptable carrier and a therapeutically effective amount of a compound according to any of the claim 1 or a pharmaceutically acceptable salt thereof.

16. A method for treating or preventing a thromboembolic disorder, comprising administering to a patient in need thereof a therapeutically effective amount of a pharmaceutical composition comprising a pharmaceutically acceptable carrier and a therapeutically effective amount of a compound according to any of the claim 1 or a pharmaceutically acceptable salt thereof.
